(19) United States
(12) Patent Application Publication
(10) Pub. No.: US 2019/0142046 A1
(43) Pub. Date: May 16, 2019

(54) FUNCTIONALLY REINFORCED DESALTED NUTRITIONAL COMPOSITIONS FROM HALOPHYTES AND PREPARATION METHOD THEREOF

(71) Applicant: PHYTO CORPORATION, Seoul (KR)

(72) Inventors: Deuk Hoi KIM, Goyang-si, Gyeonggi-do (KR); Mee Hyang KWEON, Seoul (KR); Eun Ah CHO, Seoul (KR); Hyun Joo YOON, Suwon-si Gyeonggi-do (KR); Seon Yeong PARK, Seoul (KR)

(21) Appl. No.: 16/099,070

(22) PCT Filed: Jan. 26, 2017
(86) PCT No.: PCT/KR2017/000949

§ 371 Date: Nov. 5, 2018

(30) Foreign Application Priority Data
May 4, 2016 (KR) 10-2016-0055486
Dec. 30, 2016 (KR) 10-2016-0183473

(51) Int. Cl. A23L 33/105; A23L 33/16; A23L 33/20; A23L 5/20; A61K 36/21; A61K 31/192; A61K 33/06

(52) U.S. Cl. CPC A23L 33/105; A61K 2236/331; A23L 33/20; A23L 5/23; A61K 36/21; A61K 31/192; A61K 33/06; A23V 2002/00; A23V 2200/332; A23V 2250/2132; A23V 2250/2116; A23V 2250/02; A23V 2250/16; A23V 2250/161; A23V 2250/1614; A23V 2250/1578; A23V 2300/14; A61K 2236/51; A61K 2236/53; A23L 33/16

(57) ABSTRACT

Disclosed are a functionally reinforced desalted nutritional composition, a desalted extract and a cold-water-extracted salt substitute, which are derived from halophytes that grow in coastal regions under highly saline conditions and thus retain high salt concentrations, as well as the use of the desalted nutritional composition for combating obesity. More particularly, this invention relates to a functionally reinforced desalted nutritional composition, a desalted extract and a salt substitute cold-water-extracted from halophytes that inhabit extreme environments of high salinity under high salt stress, the halophytes being desalted through a cold water extraction process at a low temperature based on the difference in water solubility of salts with change in temperature to allow only sodium chloride to be selectively removed, and the composition thus having decreased sodium content as well as having increased content of useful minerals such as potassium, as well as nutrients and physiologically active substances, which are naturally contained in halophytes.

### TECHNICAL FIELD

The present invention relates to a functionally reinforced desalted nutritional composition, a desalted extract and a cold-water-extracted salt substitute, which are derived from halophytes that grow in coastal regions under highly saline conditions and thus retain high salt concentrations, and the present invention is also concerned with the use of the desalted nutritional composition to combat obesity. More particularly, the present invention relates to a functionally reinforced desalted nutritional composition, a desalted extract and a salt substitute cold-water-extracted from halophytes that inhabit extreme environments of high salinity under high salt stress, the halophytes being desalted through a cold water extraction process at a low temperature based on the difference in water solubility of salts with change in temperature to allow only sodium chloride to be selectively removed, and the composition thus having decreased sodium content as well as having increased content of useful minerals such as potassium, as well as nutrients and physiologically active substances, which are naturally contained in halophytes.

### BACKGROUND ART

Halophytes are plants that naturally grow in saline habitats, such as in coastal regions and around salt fields, where most terrestrial plants can't survive due to the high salinity of soils. Halophytic plants, through metabolic responses that allow them to overcome salt stress, can retain high salt concentrations in their cells and tissues, and can take up seawater owing to their high osmotic potentials. When eaten, the plants taste very salty because they contain high salt concentrations. Halophytes are found growing in communities in high salt areas of salt marshes throughout the world. Representative examples of such halophytic species include Salicornia europaea, Suaeda asparagoides, and Suaeda japonica.

Salicornia europaea, which is an annual halophyte that belongs to the family Chenopodiaceae, is widely distributed in saline habitats, such as salt marshes or coastal areas, where agricultural crops generally cannot grow well, throughout the world including South Korea, Europe and North America. This plant has jointed stems, which are thick, fleshy and swollen and are deep green, and grows to 20 to 40 cm tall. This succulent herb is, in the classic manual of Chinese herbal medicine ‘Shennong Bencaojing’, referred to as ‘Hamcho’ and ‘Yeomcho’, which mean a salty herb, due to its salty taste, and is also called ‘Shincho’, which means a very rare numinous herb. In North America, the plant is known as glasswort. It is also known as ‘Samphire’ in Europe and ‘Aatkaeso Sangoso’ in Japan. Since S. europaea grows in salt marshes of high salinity, it can accumulate salts to high concentrations in its tissues so as to adjust to osmotic pressure. For this reason, glasswort powder has been used as a vegetable table salt substitute. Recent studies have revealed that, as well as sodium (Na), S. europaea retains calcium (Ca), potassium (K), magnesium (Mg) and iron (Fe) at higher levels relative to other plant species while it contains abundant amounts of essential amino acids, edible fibers, physiologically active nutrients, etc. At this point, the succulent herb has been reported to have various beneficial physiological effects, for example, anti-thrombotic, anti-diabetic, hypolipidemic, anti-hypertensive, and antioxidant effects, as well as melanin synthesis inhibition. Owing to its salty taste and various physiological effects, S. europaea has been utilized in folk medicine and has been known to be used as a medicinal herb for lifestyle-related diseases. According to the Korea Native Medicinal Herb Research Association, S. europaea has beneficial effects for circulatory and gastrointestinal systems. The Ohara Sanso Institute of incurable diseases in Japan revealed that S. europaea has excellent efficacies for several cancers, sinus infection, arthritis, hypertension, hypotension, backache, obesity, hemorrhoids, diabetes, etc. In the Japanese ancient book about medicinal herbs ‘Daehwaboncho’, S. europaea is noted as ‘Shincho’, ‘Bokcho’, which means an herb bringing good fortune, or ‘Yeomcho’, and is described as eliminating toxins and coprostasis accumulated in the bodies and to have excellent therapeutic effects for various incurable diseases such as cancer, uterine myoma and sinus infection. In addition, S. europaea improves blood circulation, strengthens blood vessels, and has therapeutic effects for both hypertension and hypotension as well as for sinus infection, nephritis, arthritis, etc. Moreover, since S. europaea is effective in treating purulent inflammation and has diverse antibacterial activities, it has been applied to treat inflammations, arthritis-induced swelling, and the like. Also, S. europaea help relieve chronic fatigue and clear the brain to concentrate the mind.

Seablite, whose botanical name is ‘Suaeda asparagoides’, is an annual halophytic plant that belongs to the family Chenopodiaceae, and is widely distributed in saline habitats such as coastal areas in South Korea, Japan, China, etc. This plant, which is also known by the name ‘Suaeda glauca’, has narrow and thin leaves like pine needles and, in South Korea, is commonly called ‘Gaetsolnamul’, which means a coastal herb having thin leaves like pine needles. S. asparagoides can be eaten, but its intake is limited owing to its high saline content and is thus merely used as a vegetable salt substitute. S. asparagoides has excellent effects of lowering fever and alleviating hypertension and poor hepatic function while degrading coprostasis and waste matter accumulated in the intestine and excreting them outside the body, thus being useful for constipation, obesity, etc. In addition, this plant contains physiologically active substances such as polyphenol compounds, and thus has antioxidant activity and inhibits the permeability of capillary vessels leading to strengthening of blood vessels, as well as having active-oxygen scavenging activity and inhibiting lipid peroxidation. Thus, when desalted, S. asparagoides has the potential to be developed into a functional food.

Suaeda japonica, which is an annual halophyte that belongs to the family Chenopodiaceae, is a salt-tolerant plant that, like S. europaea, retains large quantities of salt in its tissues and can grow well in highly saline soils. This plant inhabits South Korea, Japan, etc., grows to 20 to 50 cm tall, and is green first and turns violet-red later. This plant also can be eaten, but its intake is limited owing to its high saline contents and is thus merely used as a vegetable salt substitute. In Chinese herbal medicine, the whole part of the plant other than the roots has been used as an herbal medicine and has been known to be effective in treating fever, hypertension, dyspepsia, constipation, obesity, and the like. The plant contains large quantities of natural minerals and is rich in secondary metabolites such as polyphenols, flavonoids and saponins, which are highly bioavailable. Thus, when desalted, S. japonica has high potential to be utilized as a functional material. S. japonica has physiological activities including antioxidant activity and inhibitory activity against α-glucosidase, which is implicated in the postprandial rise in blood glucose. Some studies on components of S. japonica have revealed that this plant contains glycine betaine, which is involved in salt stress tolerance, 2′-hydroxy-6,7-methylenedioxy-isoflavone, loliolide, dehydrovomifoliol, uridine, and the like.

Meanwhile, the increased occurrence of extreme unusual weather events associated with global warming is already affecting food security. Climate change is leading to a decline in crop productivity. This and other factors are worsening the global food situation, including rising feedstuff demand caused by tremendous demand for animal food as economies grow in newly industrializing countries, such as China and India, and the usage of food resources for biofuel production. To deal with climate change and water shortage, there is growing interest in the development of seawater agriculture, which is a future core technology based on the use of sea water to secure stable food resource supplies. Some regions currently facing chronic water shortage in the world have little fresh water available even for human consumption, let alone for agricultural use. Thus, since the current agricultural production system, which depends exclusively on fresh water, has a large risk associated with water shortage, great interest has been taken in utilizing seawater. Over 97% of the water on the earth is seawater, and this huge amount of seawater can be used to alleviate drought and desertification as well as to create new food resources. In this respect, halophytic plants, which can be cultivated by seawater agriculture, may be potentially good alternatives for overcoming nutrient and food crises in situations of water and food shortage.

To date, halophytes have been known to be mainly used as food sources like salads and as vegetable table salt substitutes. Many studies have revealed that powders or extracts from halophytes have beneficial functions, but halophyte products have not been developed as functional foods or materials. This is because the high salinity of halophytes limits their utilization to use as salty sources or soybean sources.

Korean Pat. No. 10-0724705 (entitled “Edible Liquid Type Composition Comprising Glasswort (S. europaea) Extract”) discloses a method of preparing a liquid-type composition containing glasswort as an effective ingredient, including extracting raw halophyte materials including glasswort and mixing the extract with food additives and others to yield a drink, in which the drink mixture can be further dried to yield a solid. This patent also describes a food manufacturing method characterized by kneading the drinkable composition at a predetermined ratio. However, since these products are not desalted and thus have a high content of sodium chloride, their amounts when added or eaten are limited. When halophytes are ingested in sufficiently large amounts to absorb the effective ingredients therein, excessive sodium intake may increase the risk of hypertension, cardiovascular disease, or the like, thereby causing health problems.

In order to solve these problems, some studies have been conducted for eliminating salts from halophytes. Representative desalting methods are as follows.

(1) Korean Pat. No. 10-1218355 discloses a method of preparing betacyanin from red glasswort (S. europaea). The method of preparing the natural edible pigment betacyanin is based on extracting red glasswort, desalting the extract by electrodialysis, and drying the desalted extract. However, this method only serves to obtain a red pigment from glasswort that has turned red, and glasswort necessarily turns red due to physiological changes as chlorophylls are destroyed right before it withers. Also, during the electrodialysis process, some loss may occur, besides sodium salt, of minerals useful for human bodies, such as potassium, calcium, magnesium iron, and other useful low-molecular weight ingredients.

(2) Korean Laid-open Publication Pat. No. 10-2006-0110023 describes a method by which glasswort is extracted with hot water or ethanol, the extract powder then being mixed with starch paste and other ingredients to be made into pills. This method is problematic in that the hot water and ethanol extracts cannot contain all glasswort nutrient and in that the high salt concentrations of the extracts are not removed.

(3) Korean Pat. No. 10-1095619 discloses a method for lowering the salt content of glasswort and a storage method for the desalted glasswort. This method includes cutting glasswort into about 0.5 cm lengths, stirring the herb pieces in a 0.1% to 1.0% NaCl solution for 10 to 40 minutes, and storing the salt-reduced glasswort extract at 35° C. and 50° C. However, this method has the following problems: since fresh herbs are cut, immersed in a salt solution and stirred at a high temperature above room temperature for a long period of time, most organic compounds contained in glasswort, except for water-insoluble dietary fiber, are lost, and the salt solution does not ensure a strong desalting effect.

(4) Korean Pat. No. 10-1287065 discloses a method for preparing glasswort powder having improved sanitation and digestibility. This method includes washing fresh glasswort herb, extracting juice from the herb, sterilizing the glasswort juice at 90 to 110° C. for 5 to 60 minutes, heating the juice to 50 to 70° C., decompressing and concentrating the juice, degrading the spray-dried powder and remaining juice residues through enzymatic treatment, and pulverizing the resulting product. However, since this method does not include a substantial desalting process, high salt concentrations still remain in the glasswort powder.

In addition to halophytes, desalination studies have been performed on other materials, and representative efforts are as follows.

(1) Korean Pat. No. 10-1289769 describes a method for preparing desalted milk based on eliminating singly charged minerals contained in milk. In order to eliminate singly charged sodium ions from raw milk, this method includes passing raw milk through a chloride anion exchange resin and eliminating singly charged minerals through membrane separation. This method is applicable only to liquid-phase samples not containing insoluble solids, and has another problem in that milk acidity increases when milk is passed through the anion exchange resin. In addition, the anion exchange resin can absorb non-mineral organic substances, for example, essential amino acids and alkaloids, thereby causing the loss of a variety of such physiologically active ionic substances.

(2) Electrodialysis is a process of separating ionic components from a solution. This process is theoretically based on the mass transfer theory, in which ionic components in a solution are selectively passed through a cation exchange resin membrane and an anion exchange resin membrane by a voltage applied to an electric field. Also, electrodialysis is a membrane process that is most commonly used along with reverse osmosis and ultrafiltration. Such an electrodialysis process is mainly applied for desalination using an electrically charged membrane. Korean Pat. No. 10-0561103 discloses an electrodialytic method of lowering the salt concentration of Korean traditional soy sauce. In this patent, the electrodialysis process resulted in decreases in the salinity of soy sauce from 23.67% to 20.46%, 15.2% and 10.81%. However, since electrodialytic desalination requires continuous circulation of a liquid-phase sample, it is impossible to completely remove salt from a liquid. Moreover, the method cannot be applied to samples other than liquids.

(3) Like electrodialysis, ultrafiltration is unable to selectively eliminate only sodium salt, and is also disadvantageous in terms of removing useful minerals such as potassium, calcium and magnesium along with sodium. The ultrafiltration method has additional drawbacks in that low-molecular-weight organic compounds less than 200 daltons are lost in a sample and high costs are required for maintaining and managing the equipment.

(4) Osmosis is a natural process. When two solutions with different concentrations of a solute are separated by a semipermeable membrane, a solvent moves across a membrane partition from the side of low solute concentration toward the side of high solute concentration. The driving force for the movement of the solvent is the chemical potential generated by the difference in solute concentration. When the solvent moves into the more highly concentrated solution, pressure is generated and applied to the more highly concentrated solution, this pressure being called osmotic pressure. In reverse, when an external pressure higher than osmotic pressure is applied, the solvent is forced to move from the high to the low solution concentration, and this phenomenon is called reverse osmosis. The principle of reverse osmosis has been applied using a pressure gradient typically between 30 and 100 times atmospheric pressure as a driving force to remove various kinds of salts or organic substances through a semipermeable membrane, and this process is called a reverse osmosis separation process. This process is known for its use mainly in the desalination of seawater to obtain fresh water, deionized water preparation for the semiconductor industry, various industrial wastewater treatment processes, and so on. Korean Laid-open Publication No. 10-2005-0122447 describes the use of reverse osmosis for concentrating soy sauce and lowering its salt concentration. However, in this publication, since desalination is achieved using a solute concentration gradient, sodium salt alone cannot be selectively removed from a solution, and thus other useful minerals, low-molecular-weight nutrients and organic compounds are removed along with sodium. Thus, this desalination process is not applicable to halophytes.

(5) Korean Pat. No. 10-1102259 discloses a desalting method of salted and fermented food using alcohol. In this patent, desalination is achieved by adding alcohol in an amount 0.5 to 10 times more than a raw material to a salted and fermented food to lower salt solubility and thus precipitate the salt, followed by removing the salt through a physical process. With this method, a small amount of salt can be precipitated and removed. However, the alcohol addition, rather than the salt removal effect, can cause proteins to coagulate and denature and can also reduce the solubility of polysaccharides, thus leading to precipitation. In particular, a large amount of acidic polysaccharides and protein-bound polysaccharides rapidly precipitate, thus resulting in a very large loss of nutrients contained in a raw material.

In this regard, the present inventors, in order to solve the above mentioned problems of desalination, conducted thorough and intensive research into a method capable of effectively removing only sodium salt (NaCl) from halophytes without the loss of useful minerals such as potassium, calcium and iron, nutrients such as carbohydrates and proteins, and useful physiologically active substances such as chlorophylls, polyphenols and flavonoids. This research resulted in the development of a desalination method using the difference in water solubility of salts with change in temperature (see FIG. 1). In detail, when dry halophyte powder is extracted with stirring in cold water at a low temperature (9° C. or lower), as compared with the cases of using room-temperature water and hot water, useful minerals excluding sodium salt and organic soluble components were found to be eluted at remarkably low levels while there was no large difference in the elution degree of sodium salt. In addition, as compared with before desalination, the desalted powder was found to have remarkably increased content of dietary fiber as well as polyphenols, flavonoids and chlorophylls. Moreover, the desalted halophyte powder, compared with before desalination, was found to have remarkably improved activities, such as anti-oxidant, anti-thrombotic, anti-hypertensive and anti-diabetic activities. Further, a cold-water-stirred extract obtained during the halophyte desalination process, unlike conventional glasswort salts, was found to have a high content of sodium chloride and a clean salty taste having a savory (umami) flavor, and thus has the potential to be used as a 100% pure vegetable salt substitute.

Obesity is a kind of metabolic disorder that is caused by various factors such as excess energy intake, genetic susceptibility and decreased physical activity. Obesity refers to a condition that is characterized not only by excess weight but also by an increased content of body fat. In modern times, many people become obese owing to excess nutrient intake, and obesity is a growing serious socioeconomic health problem today. In past centuries, the prevalence of obesity has been rising mainly in advanced countries, but, in recent years, the population of overweight people has rapidly expanded in South Korea. Obesity has been known as a risk factor for many metabolic disorders such as cardiovascular disease, diabetes, non-alcoholic hepatitis, cancers, Alzheimer's disease and osteoarthritis, and it is thus becoming classified as a serious modern disorder. In addition, obesity increases intracellular oxidative stress, which facilitates the dysregulation of adipocytokine release from adipose tissues, which contributes to the development of several diseases, such as metabolic syndromes, including atherosclerosis and diabetes, and ischemic heart disease. Physical exercise, diet restriction, medication and surgical operations are the major preventive or therapeutic methods of treating obesity. However, anti-obesity drugs, which are chemical synthetic substances, have been known to have strong anti-obesity effects, but also have many side effects. In this regard, recently, there is growing interest in natural plant materials that are safe and have mild side effects. Representative examples of such anti-obesity natural plant materials include polyphenols, which suppress fat synthesis and adipocyte differentiation, chili pepper capsaicin, which reduces body fat by activating body energy metabolism, and vegetable dietary fiber, which inhibits fat absorption and gives a feeling of satiation.

Many previous studies have been conducted to investigate the anti-obesity efficacy of glasswort. Collectively, experiments have been carried out using an extract obtained by extracting glasswort with water or alcohol (hydrous) and non-desalted glasswort powder.

Based on the salt content contained in samples (NaCl content of hot-water extract: about 55-65%, NaCl content of alcohol (hydrous) extract: about 30-40%, NaCl content of glasswort powder: about 35-40%), the same amount of sodium chloride as that of the glasswort powder was added to a high-fat diet-induced obesity control group. The glasswort samples of the above studies were found to have an anti-obesity effect but still retain sodium chloride contained in the raw material, thus limiting their direct development into functional materials. For this reason, the glasswort samples were suggested only for use as a table salt substitute having anti-obesity efficacy (Journal of the Science of Food Agriculture, 2015, 95:3150-3159).

In contrast, in the halophyte-derived functionally reinforced desalted nutritional composition developed in the present invention, sodium chloride could be effectively removed alone from glasswort. The present inventors thus felt that they were able to overcome the problems encountered in previous studies, and investigated to determine the anti-obesity effect of the desalted nutritional composition. The functionally reinforced desalted nutritional composition was found to have remarkably excellent anti-obesity and body-fat-reducing effects compared to before desalination, ensuring potential for application as a functional food and functional feedstuff effective in preventing and/or treating obesity, thus leading to the present invention.

### DISCLOSURE

### Technical Problem

It is therefore an object of the present invention to provide a functionally reinforced desalted nutritional composition from halophytes, which has a low sodium content as well as increased content of nutrients and functional physiologically active substances naturally contained in halophytes, such as insoluble dietary fiber, carbohydrates, potassium (K), magnesium (Mg), polyphenols, flavonoids and chlorophylls, a desalted extract from halophytes, and methods thereof.

It is another object of the present invention to provide a salt substitute cold-water-extracted from halophytes and a method thereof, the salt substitute being obtained during a halophyte desalination process.

It is a further object of the present invention to provide a pharmaceutical composition and a functional food for combating obesity and for reducing body fat.

### Technical Solution

In order to accomplish the above objects, the present invention provides a method of preparing a functionally reinforced desalted nutritional composition from a halophyte, comprising the steps of (a) mixing dried powder of the halophyte with water at 9° C. or lower and stirring the mixture; (b) centrifuging the stirred mixture and removing a supernatant having a high salt content to recover a desalted precipitate; and (c) drying the desalted precipitate.

The present invention also provides a functionally reinforced nutritional composition from a halophyte, comprising sodium of 0.04 to 6.8 wt % and carbohydrates of 61 wt % or greater, based on the dry weight.

In addition, the present invention provides a method of preparing a functionally reinforced desalted extract from a halophyte, comprising the steps of (a) mixing dried powder of the halophyte with water at 9° C. or lower and stirring the mixture; (b) centrifuging the stirred mixture and removing a supernatant having a high salt content to recover a desalted precipitate; (c) extracting the desalted precipitate in a liquid phase to obtain an extract; and (d) drying the liquid-phase extract.

The method of preparing a functionally reinforced desalted extract from a halophyte is characterized in that it further comprises drying the desalted precipitate before the liquid-phase extraction step of the desalted precipitate.

Further, the present invention provides a functionally reinforced desalted extract from a halophyte, which is characterized in that is extracted from a desalted product of the halophyte and has a total salt content of less than 11.0 wt % and insoluble dietary fiber of less than 3.2 wt %, based on the dry weight.

The functionally reinforced desalted nutritional composition from a halophyte according to the present invention is characterized in that it comprises potassium (K) of 0.1 to 3.0 wt %, calcium (Ca) of 0.1 to 2.0 wt % and magnesium (Mg) of 0.1 to 1.5 wt %, based on the dry weight.

The functionally reinforced desalted extract from a halophyte according to the present invention is characterized in that it comprises polyphenols of 0.1 to 10.0 wt % and flavonoids of 0.1 to 7.0 wt %, based on the dry weight.

The halophyte-derived functionally reinforced desalted extract is characterized in that it comprises chlorophylls of 0.3 to 10.0 wt % based on the dry weight.

The halophyte-derived functionally reinforced desalted nutritional composition is characterized in that it includes trans-ferulic acid.

Still further, the present invention provides a method of preparing a cold-water-extracted salt substitute from a halophyte, comprising the steps of (a) mixing dried powder of the halophyte with water at 9° C. or lower and stirring the mixture; (b) centrifuging the stirred mixture to obtain a supernatant; (c) concentrating the supernatant and purifying the concentrate with activated carbon; and (d) spray-drying the purified concentrate.

According to the present invention, the halophyte-derived cold-water-extracted salt substitute is characterized in that it has a total salt content of 50.0 wt % or more and a salt composition in which the weight ratio of potassium to sodium (K:Na) ranges from 1:10.1 to 1:19.0.

Still further, the present invention provides a cold-water-extracted salt substitute from a halophyte characterized in that it has a total salt content of 50.0 wt % or more and a salt composition in which the weight ratio of potassium to sodium (K:Na) ranges from 1:10.1 to 1:19.0.

The halophyte-derived cold-water-extracted salt substitute according to the present invention is characterized in that it comprises glutamic acid in an amount ranging from 0.1 to 50 mg/g.

The present invention also provides a pharmaceutical composition for combating obesity and for reducing body fat, comprising the halophyte-derived functionally reinforced desalted nutritional composition or trans-ferulic acid derived from a halophyte.

The present invention further provides a functional food for combating obesity and for reducing body fat, comprising the halophyte-derived functionally reinforced desalted nutritional composition or trans-ferulic acid derived from a halophyte.

The present invention still further provides a feedstuff for combating obesity and for reducing body fat, comprising the halophyte-derived functionally reinforced desalted nutritional composition or trans-ferulic acid derived from a halophyte.

### Advantageous Effects

The method of preparing a functionally reinforced desalted nutritional composition or a desalted extract from a halophyte according to the present invention, through a cold water desalination process based on the difference in water solubility of salts with change in temperature, enables the effective removal of only sodium chloride with no loss in useful minerals such as potassium, calcium and magnesium, nutrients such as carbohydrates and proteins, and useful physiologically active substances such as chlorophylls, polyphenols and flavonoids. The removed sodium chloride solution can be utilized as a table salt substitute owing to its high contents of sodium chloride and glutamic acid.

### DESCRIPTION OF DRAWINGS

The above and other objects, features and other advantages of the present invention will be more clearly understood from the following detailed description taken in conjunction with the accompanying drawings, in which:

FIG. 1 is a flowchart showing processes of preparing a functionally reinforced desalted nutritional composition, a desalted extract and a salt substitute, which are derived from halophytes, according to the embodiments of the present invention;

FIG. 2 is a graph showing the water solubility of salts with varying temperature;

FIG. 3 is a photograph showing the appearance of Salicornia europaea powder before and after being desalted;

FIG. 4 is a photograph in which non-desalted Salicornia powder (SP), cold-water-desalted Salicornia powder (CW-DSP) and hot-water-desalted Salicornia powder (HW-DSP) were compared with each other for chlorophyll content;

FIG. 5 is a photograph showing the results of colorimetric analysis for total polyphenols, total flavonoids and total proteins of hot-water extracts of non-desalted halophyte dried powder and hot-water extracts of cold-water-desalted halophyte dried powder' according to an embodiment of the present invention;

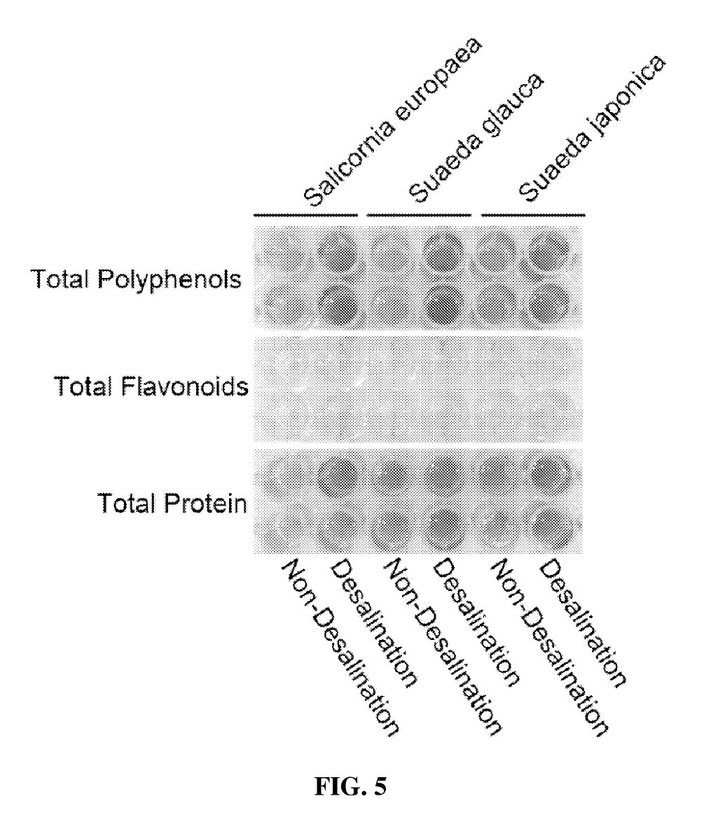

FIG. 6 is a photograph showing the results of colorimetric analysis for total sugars and total acidic sugars of hot-water extracts of non-desalted halophyte dried powder and hot-water extracts of cold-water-desalted halophyte dried powder according to an embodiment of the present invention;

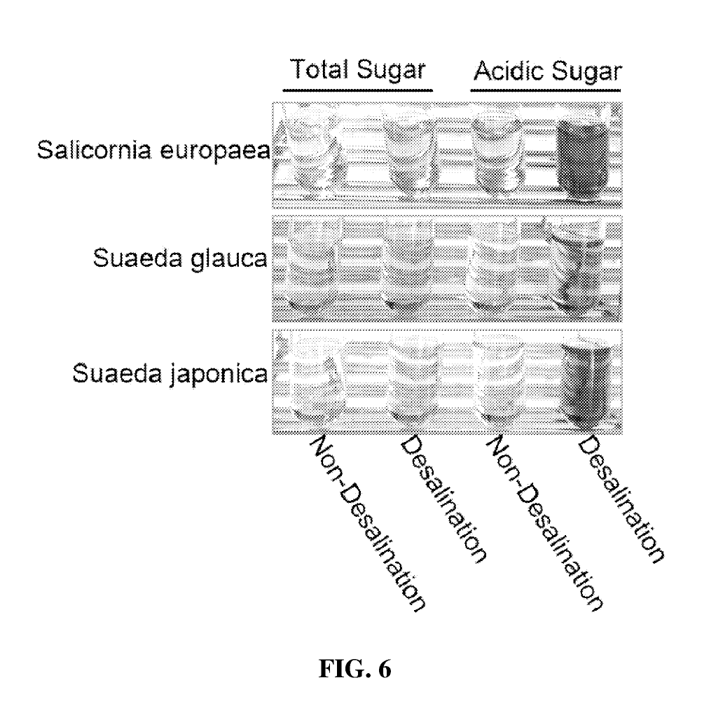

FIG. 7 is a graph showing the antioxidant activity of hot-water extracts of non-desalted halophyte dried powder and hot-water extracts of cold-water-desalted halophyte dried powder according to an embodiment of the present invention depending on concentration;

FIG. 8 is a graph showing inhibitory activity against Angiotensin-I-Converting Enzyme (ACE) of hot-water extracts of non-desalted halophyte dried powder and hot-water extracts of cold-water-desalted halophyte dried powder according to an embodiment of the present invention depending on concentration;

FIG. 9 is a graph showing inhibitory activity against alpha-glucosidase of hot-water extracts of non-desalted halophyte dried powder and hot-water extracts of cold-water-desalted halophyte dried powder according to an embodiment of the present invention depending on concentration;

FIG. 10 is a graph showing the body-weight-reducing effect of the Desalted Salicornia Powder (DSP) in obese rats induced by a high-fat diet (NC: normal control, HFD: high-fat diet (HFD)-induced obesity control group, HFD+SP200: administered with high-fat diet (HFD) plus 200 mg/kg of Salicornia Powder (SP), HFD+DSP200: administered with high-fat diet (HFD) plus 200 mg/kg of Desalted Salicornia Powder (DSP), HFD+GE200: high-fat diet (HFD) plus 200 mg/kg of Garcinia Extract (GE); mean±SD (n=10), *: p<0.05, **: p<0.01, ***: p<0.001);

FIG. 11 shows the body-fat-reducing effect of the Desalted Salicornia Powder (DSP) in obese rats induced by a high-fat diet at 6 and 12 weeks (G1: normal control group, G2: obesity control group induced by high-fat diet, G3: administered with high-fat diet plus 200 mg/kg of Salicornia Powder (SP), G4: administered with high-fat diet plus 200 mg/kg of Desalted Salicornia Powder (DSP), G5: administered with high-fat diet plus 200 mg/kg of Garcinia Extract (GE); mean±SD (n=10), *: p<0.05, **: p<0.01, #: p<0.05, ##: p<0.01);

FIG. 12 shows the abdominal-fat-reducing effect of the Desalted Salicornia Powder (DSP) in high-fat-diet-induced obese rats (NC: normal control, HFD: high-fat diet (HFD)-induced obesity control group, HFD+SP200: administered with high-fat diet (HFD) plus 200 mg/kg of Salicornia Powder (SP), HFD+DSP200: administered with high-fat diet (HFD) plus 200 mg/kg of Desalted Salicornia Powder (DSP), HFD+GE200: high-fat diet (HFD) plus 200 mg/kg of Garcinia Extract (GE); TFV: total fat volume, VFV: visceral fat volume, SFV: subcutaneous fat volume; mean±SD (n=10), *: p<0.05, **: p<0.01, #: p<0.05, ##: p<0.01);

FIG. 13 shows the results of HPLC chromatography of trans-ferulic acid, which is a marker contained in the Desalted Salicornia Powder (DSP) (A: Analytical HPLC profile of DSP-EW, B: Analytical HPLC profile of authentic trans-ferulic acid, C: Multiple preparative HPLC profile of DSP-EW; 1: caffeic acid, 2: p-coumaric acid, 3: trans-ferulic acid, 4: isorhamnetin-3-β-D-glucoside);

FIG. 14 are photographs and graphs showing the inhibitory effects of trans-ferulic acid (TFA), isolated from the Desalted Salicornia Powder (DSP), on intracellular lipid accumulation and triglyceride formation in 3T3-L1 cells (One-way ANOVA test; *: p<0.05, **: p<0.01, ***: p<0.001, #: p<0.05, ##: p<0.01, ###: p<0.001); and

FIG. 15 shows the results of Real-Time RT-PCR to determine the effect of trans-ferulic acid (TFA), isolated from the Desalted Salicornia Powder (DSP), on SREBP1, c/EBPα, PPARγ and FAS gene expression (One-way ANOVA test; *: p<0.05, **: p<0.01, ***: p<0.001, #: p<0.05, ##: p<0.01, ###: p<0.001).

### BEST MODE

Halophytes, which contain various useful substances such as dietary fiber, essential amino acids, vegetable minerals and physiologically active substances, have a limitation in their applicability owing to their high salt content. According to the present invention, based on the difference in water solubility of salts with change in temperature, when dried powder of a halophyte was extracted with stirring in cold water at a low temperature for a short time, compared with the cases of being extracted with room-temperature water and hot water, the elution of useful minerals, except for sodium, and organic soluble components was remarkably reduced, whereas there was no great difference in the elution of sodium salt.

According to the present invention, to a halophyte were added cold water, room-temperature water and hot water, respectively, followed by stirring and centrifugation to remove a supernatant. The resulting desalted extract was recovered and dried so as to yield a halophyte-derived functionally reinforced desalted nutritional composition. The extraction with cold water was found to be able to effectively remove sodium salts while minimizing the elution of organic substances.

In one aspect of the present invention, the present invention relates to a method of preparing a functionally reinforced desalted nutritional composition from a halophyte, comprising the steps of (a) mixing dried powder of the halophyte with water at 9° C. or lower and stirring the mixture, (b) centrifuging the stirred mixture and removing a supernatant having a high salt content to recover a desalted precipitate, and (c) drying the desalted precipitate, and a functionally reinforced desalted extract from a halophyte, including sodium of 0.04 to 6.8 wt % and carbohydrates of 61 wt % or greater, based on the dry weight.

Halophytes are plants that naturally grow in saline habitats, such as in coastal regions and around salt fields. Examples of halophytes include, but are not limited to, glasswort (Salicornia Spp.), Suaeda asparagoides, and Suaeda japonica.

The halophyte dried powder may be prepared by washing a halophyte to remove impurities followed by drying. A dried product itself can be used, but a powder form is preferred for more effective extraction.

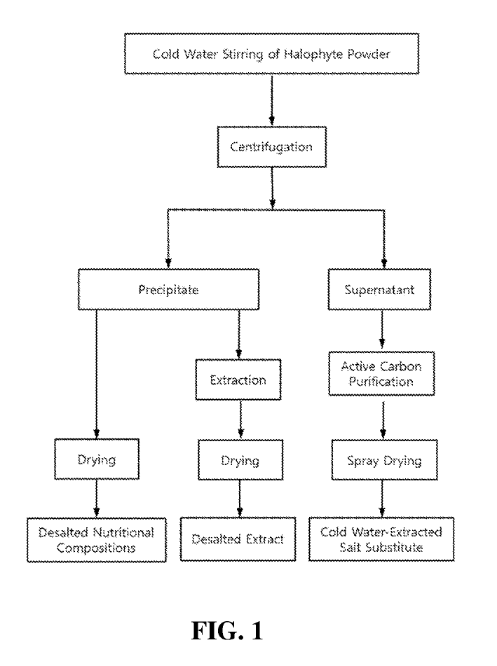

As seen in FIG. 1, the halophyte-derived functionally reinforced desalted nutritional composition is prepared as follows. First, a dried halophyte product is mixed with water at 9° C. or lower, preferably 0.1 to 4° C., and is then stirred. Preferred water is non-saline water, such as tap water or distilled water. If the stirring is carried out at a temperature of about 10° C. or higher, as compared with the condition of 0.1 to 9° C., there is no great change in the elution degree of sodium salt, but other organic soluble components and minerals, such as potassium, are eluted, causing large loss of nutrients from the desalted dried product.

The dried product of halophyte is preferably used in an amount of 40 to 70 g per 1 L for extraction. When less than 40 g is used, the solvent amount is relatively large, increasing the total amount to be centrifuged. This makes the extraction process ineffective. On the other hand, an amount greater than 70 g does not ensure effective stirring.

The stirring is preferably carried out for 1 to 5 minutes. Stirring for less than one minute results in a decrease in desalination efficiency for halophytes. When the stirring time exceeds 5 minutes, the elution of soluble organic components as well as sodium salt is increased.

After stirring, the stirred mixture is centrifuged, and a supernatant having a high salt content is then removed to recover a desalted precipitate.

The precipitate may be obtained according to a method commonly known in the art, and the method is not particularly limited as long as it can separate the stirred mixture into a supernatant and a precipitate. For example, a filtration method may be used to obtain the precipitate.

According to the intended need, the desalted precipitate may be further stirred so as to further lower the remaining small amount of salt content.

The finally desalted precipitate is recovered and dried.

Since the method of preparing a functionally reinforced desalted nutritional composition from a halophyte according to the present invention enables the effective removal of only sodium chloride with no loss of useful physiologically active substances, the desalted nutritional composition prepared according to the present invention may comprise sodium (Na) of 0.04 to 6.8 wt %, carbohydrates of 61 wt % or greater, potassium (K) of 0.1 to 3.0 wt %, calcium (Ca) of 0.1 to 2.0 wt %, magnesium (Mg) of 0.1 to 1.5 wt %, polyphenols of 0.1 to 10.0 wt %, flavonoids of 0.1 to 7.0 wt %, and chlorophylls of 0.3 to 10.0 wt %, based on the dry weight.

When the desalted precipitate, obtained by desalting a halophyte with cold water, and its dried powder were extracted with water or ethanol, as compared with an extract from a non-desalted halophyte, they were found to have remarkably reduced salt content as well as having remarkably increased content of functional components and nutrients.

In another aspect, the present invention provides a method of preparing a functionally reinforced desalted extract from a halophyte, comprising the steps of (a) mixing dried powder of the halophyte with water at 9° C. or lower and stirring the mixture; (b) centrifuging the stirred mixture and removing a supernatant having a high salt content to recover a desalted precipitate; (c) extracting the desalted precipitate in a liquid phase to obtain an extract; and (d) drying the liquid-phase extract, and a functionally reinforced desalted extract from a halophyte, which is characterized in that it is extracted from a desalted product of the halophyte and has a total salt content of less than 11.0 wt % and insoluble dietary fiber of less than 3.2 wt %, based on the dry weight.

The desalted precipitate from a halophyte is recovered using the same method described above. To elute physiologically active functional components, the desalted precipitate may be extracted with water, or may be dried and then extracted with an organic solvent, such as methanol, ethanol, butanol, ethyl acetate, acetone or diethyl ether, thus giving a desired extract. When the liquid-phase extraction is carried out with an organic solvent, the desalted precipitate is preferably dried before being subjected to liquid-phase extraction.

The liquid-phase extraction of the desalted halophyte precipitate with an organic solvent may be carried out by reflux extraction at room temperature or near a temperature at which the organic solvent becomes volatile. In this case, the desalted precipitate is preferably used in an amount of 40 to 75 g per liter of extraction solvent. An amount less than 40 g increases the cost of the extraction solvent. When the amount of solvent exceeds 75 g, extraction efficacy is reduced. The additional liquid-phase extraction process has an advantage of increasing the extract yield.

The halophyte-derived functionally reinforced desalted extract prepared according to the present invention is characterized in that it has a total salt content of less than 11.0 wt % and insoluble dietary fiber of less than 3.2 wt %, and may comprise polyphenols of 0.1 to 10.0 wt %, flavonoids of 0.1 to 7.0 wt % and chlorophylls of 0.3 to 10.0 wt %.

Since the halophyte-derived functionally reinforced desalted extract has various in vivo physiological activities, such as antioxidant, anti-thrombotic, anti-hypertensive and anti-diabetic activities, it has the potential to be applied as a raw material in foods, cosmetics, medicines, and the like.

In addition, the halophyte-derived functionally reinforced desalted nutritional composition comprises, as an effective component, trans-ferulic acid, which suppresses the adipocyte differentiation and genes involved in lipid synthesis, as well as containing dietary fiber in an amount greater than before being desalted. Thus, the composition has good anti-obesity and body-fat-reducing effects.

In yet another aspect, the present invention relates to a pharmaceutical composition and functional food and feedstuff for anti-obesity and body-fat-reducing effects, comprising the above functionally reinforced desalted halophyte dried product.

Meanwhile, after the halophyte dried powder was stirred in cold water and a desalted precipitate was recovered by centrifugation, the remaining supernatant was found to have a high content of sodium chloride while having a low content of potassium and a high content of glutamic acid, and is thus applicable as a vegetable salt substitute having a clean salty taste with a savory (umami) flavor.

Thus, in still another aspect, the present invention relates to a method of preparing a cold-water-extracted salt substitute from a halophyte, comprising the steps of (a) mixing dried powder of the halophyte with water at 9° C. or lower and stirring the mixture; (b) centrifuging the stirred mixture to obtain a supernatant; (c) concentrating the supernatant and purifying the concentrate with activated carbon; and (d) spray-drying the purified concentrate, and a halophyte-derived cold-water-extracted salt substitute prepared according to the method, which is characterized in that it has a total salt content of 50.0 wt % or more and a salt composition in which the weight ratio of potassium to sodium (K:Na) ranges from 1:10.1 to 1:19.0.

The cold-water-stirred supernatant remaining after a desalted precipitated is recovered can be concentrated to a salinity of 15 to 19% and a total solid content of 20% or higher. Thus, the supernatant may be purified using activated carbon of 3-5% based on the total solid content of the concentrate, and then spray-dried to give a halophyte-derived cold-water-extracted salt. The amount of activated carbon used in the purification may be varied to control the content of organic substances and the color of salt.

The concentration method of the cold-water-stirred supernatant is not particularly limited as long as it can concentrate the supernatant. Preferred is vacuum concentration.

### MODE FOR INVENTION

A better understanding of the present invention may be obtained through the following examples which are set forth to illustrate, but are not to be construed as limiting the present invention.

### Example 1: Evaluation of Cold Water Extraction of S. europaea for Desalting Effect

To 100 g of dried powder of glasswort (Salicornia europaea) was added 2 liters of cold water (4° C. and 9° C.), room-temperature water (20° C.) and hot water (100° C.), respectively. Extraction was carried out with stirring (300 rpm) at low temperatures (4° C. and 9° C.) and at room temperature (20° C.). Hot-water extraction was performed using a 100° C. reflux condenser.

In order to determine optimal conditions for maximizing desalination, the stirred mixture was centrifuged at a time interval of 5 min at 10,000 rpm for 20 min. Supernatants were vacuum-filtered through a membrane filter (0.45 μm pore size) and then analyzed for salinity (ATAGO ES-421, ATAGO Co. LTD. Japan) and Brix (ATAGO PAL-1, ATAGO Co. LTD. Japan). Also, the supernatants were vacuum-concentrated, freeze-dried (EYELA FDU-2200, ETELA, Japan), and then analyzed for total solid content. The measured total solid contents are, along with salt percentage in total solid and content of solids other than salt, given in Table 1, below.

TABLE 1

| | | | | | Brix | | | Solids |
|---|---|---|---|---|---|---|---|---|
| | Extraction | | Extraction | | (solid | Salt/Total | Total | other |
| Sample | temp | Water | time | Total | content)/ | solid | salt | than salt |
| (g) | (° C.) | (L) | (min) | Solid | Salinity | (%) | (g) | (g) |
| 100 | 4 | 2 | 1 | 33.0 | 1.26 | 79.4 | 26.2 | 6.8 |
| 100 | 4 | 2 | 5 | 35.0 | 1.25 | 80.0 | 28.0 | 7.0 |
| 100 | 4 | 2 | 10 | 35.6 | 1.27 | 78.9 | 28.1 | 7.5 |
| 100 | 4 | 2 | 15 | 36.3 | 1.29 | 77.7 | 28.2 | 8.1 |
| 100 | 4 | 2 | 20 | 36.8 | 1.30 | 76.9 | 28.3 | 8.5 |
| 100 | 4 | 2 | 25 | 37.2 | 1.31 | 76.3 | 28.4 | 8.8 |
| 100 | 4 | 2 | 30 | 37.5 | 1.32 | 76.0 | 28.5 | 9.0 |
| 100 | 9 | 2 | 1 | 34.3 | 1.30 | 76.6 | 26.3 | 8.0 |
| 100 | 9 | 2 | 5 | 36.4 | 1.30 | 76.9 | 28.0 | 8.4 |
| 100 | 9 | 2 | 10 | 37.0 | 1.31 | 76.2 | 28.2 | 8.8 |
| 100 | 9 | 2 | 15 | 37.8 | 1.33 | 75.0 | 28.3 | 9.5 |
| 100 | 9 | 2 | 20 | 38.3 | 1.35 | 74.2 | 28.4 | 9.9 |
| 100 | 9 | 2 | 25 | 38.7 | 1.36 | 73.7 | 28.5 | 10.2 |
| 100 | 9 | 2 | 30 | 39.0 | 1.36 | 73.3 | 28.6 | 10.4 |
| 100 | 20 | 2 | 1 | 40.3 | 1.52 | 65.8 | 26.5 | 13.8 |
| 100 | 20 | 2 | 5 | 42.3 | 1.51 | 66.2 | 28.0 | 14.3 |
| 100 | 20 | 2 | 10 | 45.8 | 1.63 | 61.5 | 28.2 | 17.6 |
| 100 | 20 | 2 | 15 | 47.3 | 1.67 | 59.8 | 28.3 | 19.0 |
| 100 | 20 | 2 | 20 | 48.8 | 1.71 | 58.4 | 28.5 | 20.3 |
| 100 | 20 | 2 | 25 | 50.3 | 1.76 | 56.9 | 28.6 | 21.7 |
| 100 | 20 | 2 | 30 | 51.0 | 1.78 | 56.3 | 28.7 | 22.3 |
| 100 | 100 | 2 | 1 | 44.1 | 1.53 | 65.3 | 27.1 | 14.9 |
| 100 | 100 | 2 | 5 | 43.6 | 1.56 | 64.0 | 27.9 | 15.7 |
| 100 | 100 | 2 | 10 | 48.0 | 1.71 | 58.5 | 28.1 | 19.9 |
| 100 | 100 | 2 | 15 | 52.8 | 1.87 | 53.6 | 28.3 | 24.5 |
| 100 | 100 | 2 | 20 | 55.3 | 1.94 | 51.5 | 28.5 | 26.8 |
| 100 | 100 | 2 | 25 | 56.7 | 1.98 | 50.6 | 28.7 | 28.0 |
| 100 | 100 | 2 | 30 | 58.5 | 2.02 | 49.4 | 28.9 | 29.6 |

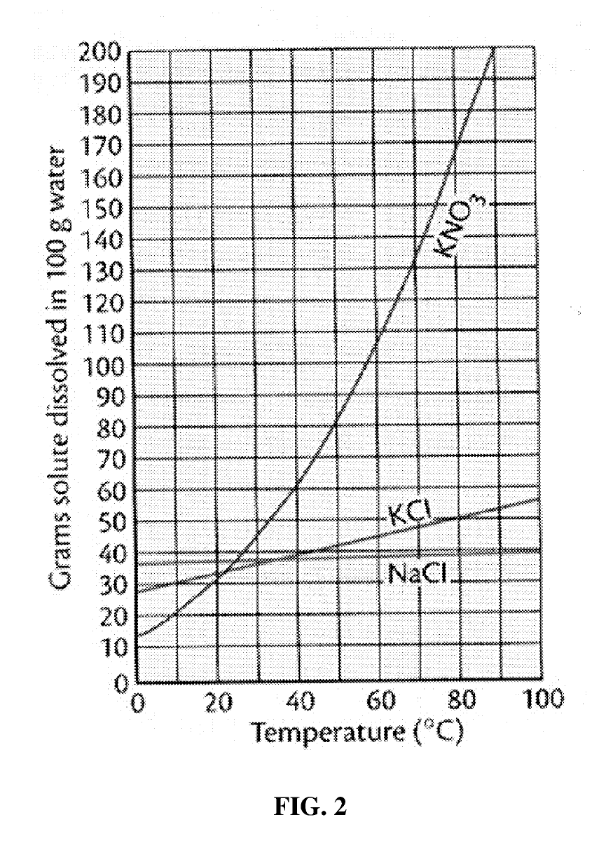

As shown in Table 1, it was found that there was almost no difference in the elution degree of salts with change in temperature. For reference, FIG. 2 shows the water solubility of salts with varying temperatures. The results shown in Table 1 correspond with the constant water solubility of NaCl according to temperature, as shown in FIG. 2.

When 100 g of dried powder of glasswort (S. europaea) was extracted with water (2 L) (4° C., 9° C., 20° C. and 100° C.) for 30 min, the eluted amounts of salts were also found to be almost the same at all test temperatures (28.5 g, 28.6 g, 28.7 g and 28.9 g, respectively). Thus, all salts contained in S. europaea were thought to be eluted within 30 min.

In contrast, the elution of soluble organic solids other than salts was found to increase greatly with increasing temperature. After extraction for 30 min, the solids were found to be eluted 2.47 times higher at room temperature (20° C.) and 3.28 times higher at 100° C. than when extracted at 4° C.

As an ideal indicator of the desalting effect, the soluble solid content (Brix, %)/salinity (%) index was measured. That is, the lower the Brix/salinity ratio, the lower the presumed loss of organic solids due to desalination. The index was found to increase gradually over time under all temperature conditions.

In addition, with regard to the difference in the Brix/salinity index, cold water extraction at 0.1 to 9° C. resulted in low index values ranging from 1.26 to 1.36; i.e. a slight difference was found. In contrast, when the extraction was carried out at 20° C. or over, high index values of over 1.5 were measured while the index ranged from 1.5 to 2.02, and the index was also found to increase gradually with increasing temperature. Thus, it is preferable that the extraction for desalination be carried out at 9° C. or lower for as short a time as possible. That is, these results indicate that, when extraction is conducted with cold water at 4° C. or lower for 4 min or shorter, the elution of organic substances is minimized while salts are effectively removed.

### Example 2: Preparation of Desalted Nutritional Compositions from S. europaea, S. asparagoides and S. japonica

Desalted nutritional compositions were prepared using three plants, Salicornia europaea, Suaeda asparagoides and Suaeda japonica, which are known to be extreme halophytes that naturally grow in South Korea. Fresh plants were washed with tap water and freeze-dried to be powdered. Based on the results from Example 1, in which, when extraction is conducted with cold water at 4° C. or lower for 4 min or shorter, the extraction of organic substances is minimized while salts are effectively removed, 100 g of the dried powder was added to 2 L of cold water (4° C.), stirred at 4° C. for 4 min, and centrifuged at 10,000 rpm for 20 min. Then, a supernatant having a high salt content was removed, and a desalted precipitate was recovered. The recovered precipitate was desalted once more by the same method as above so as to minimize remaining sodium salt. The precipitates thus obtained were freeze-dried to give halophyte-derived desalted nutritional compositions (desalted powder).

### Test Example 1: Analysis for Components of Halophyte-Derived Desalted Nutritional Compositions

Non-desalted halophyte dried powders from S. europaea, S. asparagoides and S. japonica and the desalted nutritional compositions (desalted powder), prepared in Example 2 from the same halophytic species, were analyzed for their contents of sodium, nutrients and functional components, and the results are given in Table 2, below. Analysis for calorie carbohydrate and protein content was performed according to the general analytical method of the Korean Food Standards Codex (Korean Food Industry Association). Sodium, potassium, magnesium, iron and calcium levels were measured through wet analysis using an acidic digestion method using nitric acid and then by Inductively Coupled Plasma Spectrometry (ICPS).

The amounts of other components, namely polyphenols, flavonoids and chlorophylls, were determined as follows.

### 1-1: Analysis for Total Polyphenol Content

The total polyphenol content was determined in a 96-well microplate according to a modified Folin-Davis method. Non-desalted and desalted halophyte powders were extracted with 70% methanol, dried and dissolved in distilled water. 20 μl of each sample was mixed with 250 μl of 2% sodium carbonate and 15 μl of 50% Folin-Ciocalteu reagent (Sigma Co., USA), and the solution was allowed to react at room temperature for 30 min. Then, the absorbance was measured at 725 nm using a microreader (Bio-RAD, x-Mark, USA). As a standard, tannic acid solutions of 0 to 500 μg/mL (Sigma Co., USA) were used instead of the samples, and, from the calibration curve thus obtained, the amount of total polyphenols contained in the extraction samples were calculated.

### 1-2: Analysis for Total Flavonoid Content

The total flavonoid content was determined in a 96-well microplate according to a modified Abdel-Hameed method. Non-desalted and desalted halophyte powders were extracted with 70% methanol, dried and dissolved in distilled water. To 30 μl of each sample were added 200 μl of 90% diethylene glycol and 5 μl of 1 N NaOH. The solution was allowed to react at 37° C. for 1 hr. Then, the absorbance was measured at 420 nm using a microreader (Bio-RAD, x-Mark, USA). As a standard, rutin solutions of 0 to 500 μg/mL (Sigma Co., USA) were used instead of the samples, and, from the calibration curve thus obtained, the amount of total flavonoids contained in the extraction samples were calculated.

### 1-3: Analysis for Total Chlorophyll Content

1 g of each of non-desalted and desalted halophyte powders was extracted with 50 mL of 80% acetone at room temperature until the color disappeared. Then, the supernatant was isolated, and the absorbance was measured at 645 nm and 663 nm using a microreader (Bio-RAD, x-Mark, USA). The concentrations of chlorophyll a, chlorophyll b and total chlorophyll were calculated using the equations below.

Chlorophyll a (mg/mL)=12.720D663−2.580D645

Chlorophyll b (mg/mL)=25.880D645−5.500D663

Total chlorophyll (mg/mL)=7.220D663+20.30D645

TABLE 2

| Halophyte | S. europaea | | S. asparagoides | | S. japonica | |
|---|---|---|---|---|---|---|
| Powder | Before | After | Before | After | Before | After |
| Nutrients | desalting | desalting | desalting | desalting | desalting | desalting |
| Calorie | 151.63 | 224.89 | 159.84 | 217.62 | 143.69 | 201.11 |
| (Kcal/100 g) | | | | | | |
| Total | 37.98 | 74.44 | 39.41 | 73.18 | 32.25 | 67.20 |
| carbohydrates | | | | | | |
| (%) | | | | | | |
| Proteins (%) | 9.79 | 13.84 | 9.12 | 12.79 | 8.97 | 12.71 |
| Sodium (%) | 13.96 | 1.34 | 12.15 | 1.26 | 12.77 | 1.29 |
| Potassium (%) | 2.14 | 3.56 | 1.99 | 3.02 | 2.03 | 3.09 |
| Calcium (%) | 0.41 | 0.64 | 0.51 | 0.78 | 0.60 | 0.91 |
| Magnesium (%) | 0.74 | 0.99 | 0.63 | 0.81 | 1.02 | 1.33 |
| Iron (%) | 0.007 | 0.012 | 0.012 | 0.020 | 0.061 | 0.077 |
| Total | 6.8 | 11.8 | 5.9 | 10.6 | 6.6 | 10.9 |
| Polyphenols | | | | | | |
| (mg/g) | | | | | | |
| Total flavonoid | 3.3 | 5.8 | 2.5 | 5.0 | 3.4 | 5.3 |
| (mg/g) | | | | | | |
| Total | 29.49 | 54.25 | 25.63 | 45.85 | 15.71 | 32.09 |
| chlorophyll | | | | | | |
| (mg/g) | | | | | | |

As shown in Table 2, compared with before being desalted, the desalted halophyte samples were found to have increased contents of carbohydrates and crude proteins. The main component removed during desalination was found to be sodium (Na), while the concentrations of other minerals, such as potassium, calcium, magnesium and iron, were increased after desalting.

In addition, the concentrations of polyphenols, flavonoids and chlorophylls, which are expected to have useful physiological activities in halophytes, were greatly increased after desalting. These results indicate that the desalted halophyte powder is applicable as a functional nutritional composition having increased content of useful physiological active substances.

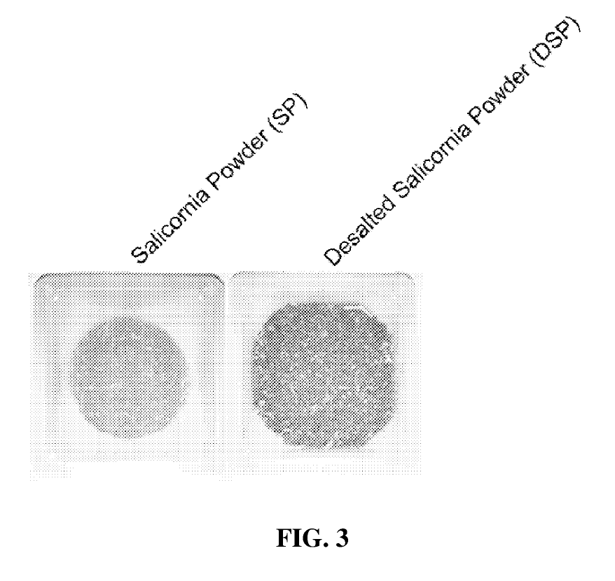

FIG. 3 shows the appearances of Salicornia europaea powder (5 g) before and after being desalted. As shown in FIG. 3, when Salicornia powder was desalted in cold water for a short time, chlorophylls were eluted only in small amounts, with almost all remaining, while the desalted powder became lightened by desalination and its volume was thus increased.

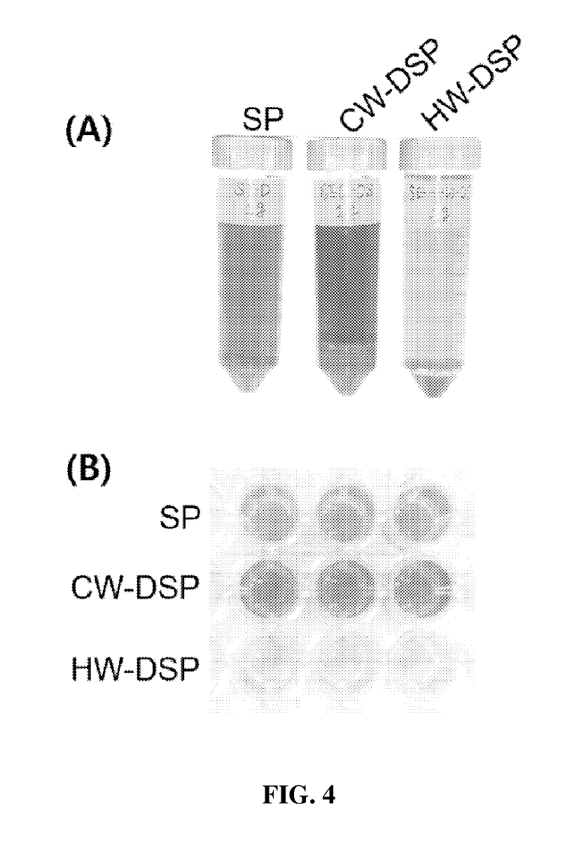

Non-desalted Salicornia powder (SP), cold-water-desalted Salicornia powder (CW-DSP) and hot-water-desalted Salicornia powder (HW-DSP) were compared with each other for chlorophyll content, and the result is shown in FIG. 4. Chlorophyll is a green pigment that is plentiful in the chloroplasts of plants in which photosynthesis occurs, and is weakly associated with proteins. Chlorophyll has a unique chemical structure that has a porphyrin (tetrapyrrole) ring with a magnesium atom at its center, and is a hydrophobic compound having a long hydrocarbon tail attached to the porphyrin ring (Rudiger, W. and Schoch, S., “Chlorophylls”, In: Plant Pigments, 1988. Academic Press, London). Due to this structural feature, chlorophyll is thought not to be eluted but to remain during desalination with cold water. Thus, the cold-water-desalted Salicornia powder (CW-DSP) was found to have chlorophyll content higher than that of the non-desalted Salicornia powder (SP).

Since chlorophyll, which is a functional raw material designated in the Health Functional Food Code, is a functional substance for improving antioxidant activity and immunity, cold-water-extracted halophyte powder has the potential to be used as a functionally reinforced nutritional composition. In contrast, since chlorophyll is weakly resistant to heat and thus easily degraded by heat, hot-water-desalted halophyte powder has a remarkably low chlorophyll content.

### Example 3: Preparation of Functionally Reinforced Desalted Extract from Halophytes (Hot-Water Extracts and Ethanol Extracts)

100 g of the functionally reinforced desalted nutritional compositions (desalted powders) from S. europaea, S. asparagoides and S. japonica, prepared in Example 2, were each added to 2 L of distilled water, reflux-extracted at 100° C. for 2 or 4 hrs, centrifuged, filtered, concentrated under pressure, and freeze-dried, thus giving halophyte-derived functionally reinforced desalted hot-water extracts.

100 g of the functionally reinforced desalted nutritional compositions (desalted powders) from S. europaea, S. asparagoides and S. japonica, prepared in Example 2 were each added to 2 L of 95% ethanol, reflux-extracted at 75±1° C. for 2 or 4 hrs, cooled to room temperature, and centrifuged. The supernatants were filtered and concentrated under pressure and freeze-dried, thus giving halophyte-derived functionally reinforced desalted ethanol extracts.

### Comparative Example 1: Preparation of Hot-Water Extracts and Ethanol Extracts from Non-Desalted Halophytes

Hot-water extracts and ethanol extracts obtained from 2 hrs extraction were prepared according to the same method as in Example 3 except that non-desalted halophytes (S. europaea, S. asparagoides and S. japonica) were used instead of the functionally reinforced desalted nutritional compositions (desalted powders) from S. europaea, S. asparagoides and S. japonica.

### Test Example 2: Evaluation for Components of Hot-Water Extracts and Ethanol Extracts

For the samples extracted for 2 hrs in Example 3 and Comparative Example 1, total sugar (carbohydrates) was measured using a modified phenol-sulfuric acid method (Kweon et. al., 1996. Agric. Chem. Biotech. 39. 15-164), and acidic sugars were measured using an m-hydroxybiphenyl method (Blumenkrantz et. al., 1973. Analytical Biochem. 54. 484-489). Total polyphenols, total flavonoids and total chlorophylls were measured according to the same method as in Test Example 1, and this test was replicated three times. The results of analysis of components of the hot-water extracts and the ethanol extracts after halophytes before and after being desalted are shown in Tables 3 and 4.

TABLE 3

| Hot-water | S. europaea | | S. asparagoides | | S. japonica | |
|---|---|---|---|---|---|---|
| extracts from | Comparative | Example | Comparative | Example | Comparative | Example |
| halophytes | Example 1 | 3 | Example 1 | 3 | Example 1 | 3 |
| Total | 33.3 | 63.0 | 25.8 | 51.0 | 26.8 | 51.4 |
| carbohydrates | | | | | | |
| (%) | | | | | | |
| Insoluble | 2.1 | 3.2 | 1.7 | 2.9 | 1.6 | 2.8 |
| dietary fiber | | | | | | |
| (%) | | | | | | |
| Total neutral | 15.5 | 30.2 | 14.2 | 24.9 | 13.0 | 25.6 |
| sugars (%) | | | | | | |
| Total acidic | 17.8 | 32.8 | 11.6 | 26.1 | 12.9 | 22.2 |
| sugars (%) | | | | | | |
| Total proteins | 7.8 | 15.9 | 6.9 | 14.0 | 5.9 | 12.1 |
| (%) | | | | | | |
| Total salts (%) | 55.8 | 5.5 | 62 | 6.0 | 58 | 5.8 |
| Total | 23.1 | 40.8 | 16.6 | 32.9 | 21.2 | 38.8 |
| polyphenols | | | | | | |
| (mg/g) | | | | | | |
| Total | 14.5 | 27.7 | 11.2 | 21.5 | 17.8 | 31.0 |
| flavonoids | | | | | | |
| (mg/g) | | | | | | |
| Total | 10.6 | 21.8 | 9.9 | 18.5 | 8.5 | 15.2 |
| chlorophylls | | | | | | |
| (mg/g) | | | | | | |

As shown in Table 3, the hot-water extracts of Comparative Example 1 from non-desalted halophytes (S. europaea, S. asparagoides and S. japonica) were found to have total salts of 55.8 to 62.0%, total carbohydrates of 25.8 to 33.3% and insoluble dietary fiber of 1.6 to 2.1%. Also, the acidic sugar contents were found to range from 11.6 to 17.8%, which are relatively high compared to other general plants, indicating that acidic sugars were composed mainly of glucuronic acid and galacturonic acid.

As compared with Comparative Example 1 (the hot-water extracts of dried powder before being desalted), the hot-water extract of the cold-water-desalted dried powder, prepared in Example 3, showed a remarkable decrease of more than about 90% in total salt content while showing remarkable increases in total sugar content (51.0 to 63.0%), and, in particular, in total acidic sugar content (22.2 to 32.8%). Many studies have reported that, among sugars, acidic sugars in particular have good immune-enhancing, anti-coagulant, anti-thrombotic, and anticancer activities. Thus, the hot-water extracts of halophyte powder obtained through desalting with cold water can be used in functionally reinforced nutritional compositions because they contain high concentrations of acidic sugars. Compared with Comparative Example 1 (the hot-water extracts of dried powder before being desalted), the hot-water extracts of Example 3 showed increases of 50 to 100% in concentrations of total polyphenols (up to 40.8 mg/g), total flavonoids (up to 31.0 mg/g) and total proteins (up to 15.9 wt %). In case of 4 hrs of hot water extraction, the hot water extracts obtained from the three desalted halophytes showed higher total salt content (9.6 to 10.8%) than 2 hrs hot water-extracted samples.

TABLE 4

| | S. europaea | | S. asparagoides | | S. japonica | |
|---|---|---|---|---|---|---|
| Ethanol extracts | Comparative | Example | Comparative | Example | Comparative | Example |
| from halophytes | Example 1 | 3 | Example 1 | 3 | Example 1 | 3 |
| Total | 16.3 | 32.0 | 18.0 | 36.3 | 17.2 | 35.8 |
| carbohydrates | | | | | | |
| (%) | | | | | | |
| Insoluble | 0.17 | 0.25 | 0.14 | 0.26 | 0.18 | 0.31 |
| dietary fiber | | | | | | |
| (%) | | | | | | |
| Total neutral | 6.5 | 14.2 | 10.2 | 21.9 | 10.3 | 25.6 |
| sugars (%) | | | | | | |
| Total acidic | 8.8 | 16.8 | 6.6 | 12.1 | 5.9 | 10.8 |
| sugars (%) | | | | | | |
| Total proteins | 14.2 | 21.5 | 14.9 | 20.0 | 15.6 | 22.5 |
| (%) | | | | | | |
| Total salts (%) | 35.4 | 3.5 | 30.6 | 2.9 | 32.1 | 3.2 |
| Total | 45.1 | 78.8 | 38.6 | 66.7 | 41.2 | 70.4 |
| polyphenols | | | | | | |
| (mg/g) | | | | | | |
| Total | 33.5 | 56.4 | 24.4 | 42.6 | 29.5 | 45.4 |
| flavonoids | | | | | | |
| (mg/g) | | | | | | |
| Total | 48.7 | 80.2 | 45.1 | 75.6 | 41.8 | 73.3 |
| chlorophylls | | | | | | |
| (mg/g) | | | | | | |

As shown in Table 4, the ethanol extracts of Comparative Example 1 from non-desalted halophytes (S. europaea, S. asparagoides and S. japonica) were found to have significantly high total salts of 30.6 to 35.4%, total carbohydrates of 16.3 to 18% and insoluble dietary fiber of 0.14 to 0.18%. Also, total neutral sugar content was found to range from 6.5 to 10.3% while total acidic sugar content ranged from 5.9 to 8.8%, all of which were lower than those of the hot-water extracts (see, Table 3).

As compared with Comparative Example 1 (the ethanol extracts of dried powder before being desalted), the ethanol extract of the cold-water-desalted dried powder, prepared in Example 3, showed a remarkable decrease of more than about 90% in total salt content while showing remarkable increases in concentrations of total neutral sugars and total acidic sugars. In particular, the ethanol extracts were found to contain plenty of polyphenols, flavonoids and chlorophylls compared with the hot-water extracts, and, compared with Comparative Example 1 (the ethanol extracts of dried powder before being desalted), showed remarkable increases in the content of total polyphenols (76.7 to 90.8 mg/g), total flavonoids (52.6 to 66.4 mg/g) and total chlorophylls (85.3 to 98.2 mg/g). In case of 4 hrs of ethanol extraction, the ethanol extracts obtained from the three desalted halophytes showed higher total salt content (5.5 to 6.9%) than 2 hrs ethanol-extracted samples.

These results demonstrated that the methods of preparing a functionally reinforced desalted nutritional composition and a desalted extract from a halophyte according to the present invention, through a cold water desalination process based on the difference in the water solubility of salts with change in temperature, enables the effective removal of only sodium chloride without causing useful functional plant compounds to be eluted, leading to a remarkable increase in their content compared to the case of not being desalted. Thus, the functionally reinforced desalted nutritional composition and the desalted extract from a halophyte according to the present invention have the potential to be applied as a functionally reinforced good nutritional material.

### Test Example 3: Evaluation for Pharmaceutical Activities of Hot-Water Extracts from Halophytes

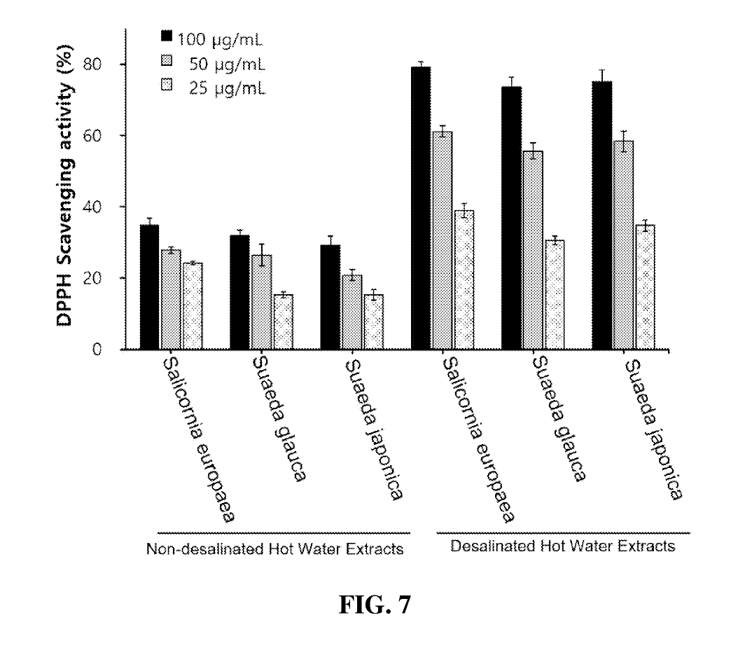

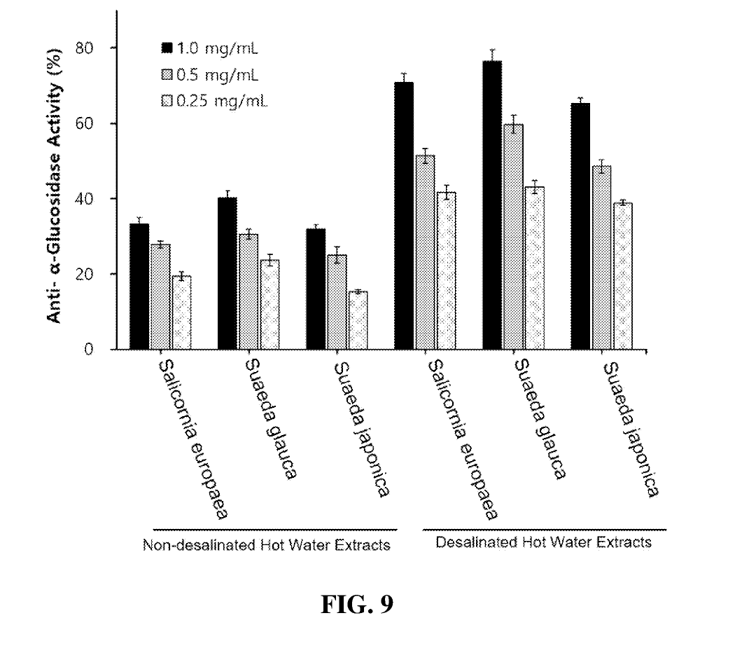

The hot-water extracts (samples) of halophytes before and after being desalted, prepared in Comparative Example 1 and Example 3, were evaluated for antioxidant, anti-thrombotic, angiotensin-I-converting enzyme (ACE)-inhibiting and α-glucosidase-inhibiting activities. The test was replicated three times, and the results are given in Table 5 and in FIGS. 7 to 9.

### 3-1: Antioxidant Activity

Antioxidant activity was assayed using 1,1-diphenyl-2-picryl hydrazyl (DPPH, Sigma Co., USA) based on the method of Blois (Chen, et. al., 1999. J. Agric. Food Chem. 47. 2226-2228).

In brief, 4 mg of DPPH was dissolved in 50 ml of ethanol, and 180 μl of the resulting DPPH solution was added to a 96-well microplate. Then, each sample was added at various concentrations (25, 50 and 100 μg/ml), mixed for 5 sec, and allowed to react at room temperature for 20 min. The reduction of the DPPH radical, relative to a control not containing a sample, was determined by reading the absorbance at 517 nm. The free radical scavenging activity was expressed as the inhibition percentage of free radicals by the sample. The IC₅₀ value is defined as the sample concentration required to scavenge 50% of DPPH free radical.

When reactive oxygen species (ROS) and free radicals generated in cells by our own metabolism are excessively produced, they cause oxidative stress in respective parts of our bodies and thus make it difficult to maintain intracellular homeostasis, eventually leading to a wide variety of diseases, including cancer, brain diseases, such as stroke and Parkinson's disease, heart diseases, ischemia, arteriosclerosis, skin diseases, digestive diseases, inflammation, rheumatoid arthritis, autoimmune disease, and aging. Thus, antioxidant compounds, which remove reactive oxygen species or inhibit free-radical production, can be used for preventing and/or treating various diseases and for suppressing skin aging, which are caused by intracellular oxidative stress.

Many anti-oxidative polyphenolic and flavonoid compounds have been already reported to be isolated from halophytic plants including S. europaea. As shown in FIG. 7, the samples prepared in Example 3 (the cold-water-desalted hot-water extracts), compared to the samples of Comparative Example 1 (the non-desalted hot-water extracts), were found to have stronger antioxidant activity (at 100 μg/ml, the antioxidant activity was increased about 2.3 times from 34.7% (the hot-water extract from non-desalted Salicornia europaea) up to 79.17% (the hot-water extract from S. europaea desalted in cold water)). This result significantly correlates with the changes in the content of total polyphenols and flavonoids present in the hot-water extracts of halophyte powder before and after being desalted in cold water.

### 3-2: Anti-Thrombotic Activity

Anti-thrombotic activity was evaluated by assaying anti-coagulant activity using a previously known method (Sohn et al., 2004. Kor. J. Pharmacogn 35. 52-61; Kwon et al., 2004. J. Life Science, 14. 509-513; Ryu et al., 2010. J. Life Science, 20. 922-928), and Prothrombin Time (PT) and activated Partial Thromboplastin Time (aPTT) were measured. Used was commercially available control plasma (MD Pacific Technology Co., Ltd, Huayuan Industrial Area, China), and PT and aPTT levels were measured as follows.

### 3-2-1: Prothrombin Time (PT)

30 μl of control plasma (MD Pacific Co., China) and 5 μl of concentrations (2.5 and 5.0 mg/ml) of each sample were added into a test cuvette of a Genius Semi-automatic Coagulometer CA 51-52 (Shenzhen, China). The cuvette was allowed to warm at 37° C. for 3 min, and 40 μl of PT reagent (Diagon, Hungary) was added. Then, the clotting time was recorded. The mean clotting time of four replicates was calculated. As a positive control, aspirin (Sigma Co., USA) was used, and, instead of a sample, DMSO was used as a solvent control. DMSO exhibited a clotting time of 18.1 sec. Prothrombin inhibitory activity was expressed as the clotting time of a sample (Ts) divided by the clotting time of the solvent control (Tc), that is, Ts/Tc, and the results are given in Table 5, below.

3-2-2: Activated Partial Thromboplastin Time (aPTT)

30 μl of plasma and 5 μl of concentrations (2.5 and 5.0 mg/ml) of each sample were added into a test cuvette of a Genius Semi-automatic Coagulometer CA 51-52 (Shenzhen, China). The cuvette was allowed to warm at 37° C. for 3 min, 20 μl of aPTT reagent (Diagon, Hungary) was added, and the cuvette was allowed again to stand at 37° C. for 3 min. 20 μl of CaCl₂) (35 mM) was added and the clotting time was recorded. Instead of a sample, DMSO was used as a solvent control, and exhibited a clotting time of 58.0 sec. The mean clotting time of four replicates was calculated. Inhibitory activity against coagulation factors was expressed as the clotting time of a sample (Ts) divided by the clotting time of the solvent control (Tc), that is, Ts/Tc, and the results are given in Table 5, below.

TABLE 5

| | | | PT | | aPTT | |
|---|---|---|---|---|---|---|
| Hot-water | | | Clotting | | Clotting | |
| extract from | | Conc. | time | | time | |
| halophyte | Samples | (mg/mL) | (sec) | Ts/Tc | (sec) | Ts/Tc |
| S. europaea | Comparative | 2.5 | 18.30 ± 0.05 | 1.01 ± 0.04 | 60.80 ± 3.04 | 1.05 ± 0.03 |
| | Example 1 | 5.0 | 19.60 ± 0.59 | 1.08 ± 0.04 | 70.10 ± 3.51 | 1.21 ± 0.04 |
| | Example 3 | 2.5 | 28.70 ± 0.86 | 1.59 ± 0.06 | 88.30 ± 4.42 | 1.52 ± 0.05 |
| | | 5.0 | 33.50 ± 1.01 | 1.85 ± 0.07 | 128.90 ± 6.45 | 2.22 ± 0.07 |
| S. | Comparative | 2.5 | 18.46 ± 0.55 | 1.02 ± 0.04 | 58.58 ± 2.93 | 1.01 ± 0.03 |
| asparagoides | Example 1 | 5.0 | 20.27 ± 0.61 | 1.12 ± 0.05 | 60.9 ± 3.05 | 1.05 ± 0.04 |
| | Example 3 | 2.5 | 26.79 ± 0.80 | 1.48 ± 0.07 | 81.78 ± 4.09 | 1.41 ± 0.05 |
| | | 5.0 | 32.04 ± 0.96 | 1.77 ± 0.04 | 106.14 ± 5.31 | 1.83 ± 0.03 |
| S. japonica | Comparative | 2.5 | 17.91 ± 0.54 | 0.99 ± 0.03 | 56.84 ± 2.84 | 0.98 ± 0.03 |
| | Example 1 | 5.0 | 18.46 ± 0.55 | 1.02 ± 0.10 | 64.96 ± 3.25 | 1.12 ± 0.04 |
| | Example 3 | 2.5 | 23.89 ± 0.72 | 1.32 ± 0.05 | 80.62 ± 4.03 | 1.39 ± 0.05 |
| | | 5.0 | 29.68 ± 0.89 | 1.64 ± 0.07 | 94.54 ± 4.73 | 1.63 ± 0.09 |

Notes:
Solvent control: PT = 18.1 sec, aPTT = 58.0 sec
Positive control (aspirin (1.5 mg/mL)); PT = 21.18 sec, aPTT = 77.28 sec

The blood, which is a body constituent, has a wide variety of critical functions, such as transporting oxygen, nutrients and wastes, acting as a buffer, maintaining body temperature, regulating osmotic pressure, maintaining ion balance, keeping water content constant, humoral regulation, maintaining and regulating blood pressure, and protecting the body. Under normal blood circulation, the blood clotting system and the clot dissolution system are regulated in a mutually complementary manner to facilitate blood circulation. The normal blood clotting process occurs as follows. Platelets adhere to the wall of the damaged blood vessels and aggregate, promoting the formation of a platelet plug (primary clot). Then, the blood clotting system is activated, and a fibrin clot is formed around platelet clumps. Substances inhibiting the thrombin activity can be used to prevent and treat various clotting disorders caused by abnormal excessive blood clotting. Also, the intrinsic coagulation pathway lead to the formation of a fibrin clot. The intrinsic pathway is activated by a sequential activation of coagulation factors, XII, XI, IX and X, in stepwise order to convert prothrombin to active thrombin. The specific inhibition of clotting factors is also a major target in developing therapeutic agents for clotting disorders.

As shown in Table 5, as compared with the control, the S. europaea sample prepared in Comparative Example 1 (the non-desalted hot-water extracts) displayed, at 5 mg/ml, slightly increased PT and aPTT values, 1.08 times and 1.21 times, respectively, whereas the S. europaea sample prepared in Example 3 (the cold-water-desalted hot-water extracts), at the same concentration, was found to have considerably prolonged PT and aPTT, 1.85 times and 2.22 times increased, respectively, indicating excellent anti-thrombotic activity.

### 3-3: ACE Inhibitory Activity

Inhibitory activity on Angiotensin-I-Converting Enzyme (ACE) was measured by a slightly modified Cushman and Cheung method. 25 μl of various concentrations (0.25, 0.5 and 1.0 mg/ml) of each sample was mixed with 25 μl of an ACE (2.5 unit) supernatant, which was prepared by dissolving 1 g of rabbit lung acetone powder (Sigma Co., USA) in 10 ml of 0.1 M sodium borate buffer containing 0.3 M NaCl, and 50 μl of 0.1 M sodium borate buffer (pH 8.3) containing 0.3 M NaCl. Then, the mixture was preincubated at 37° C. for 10 min.

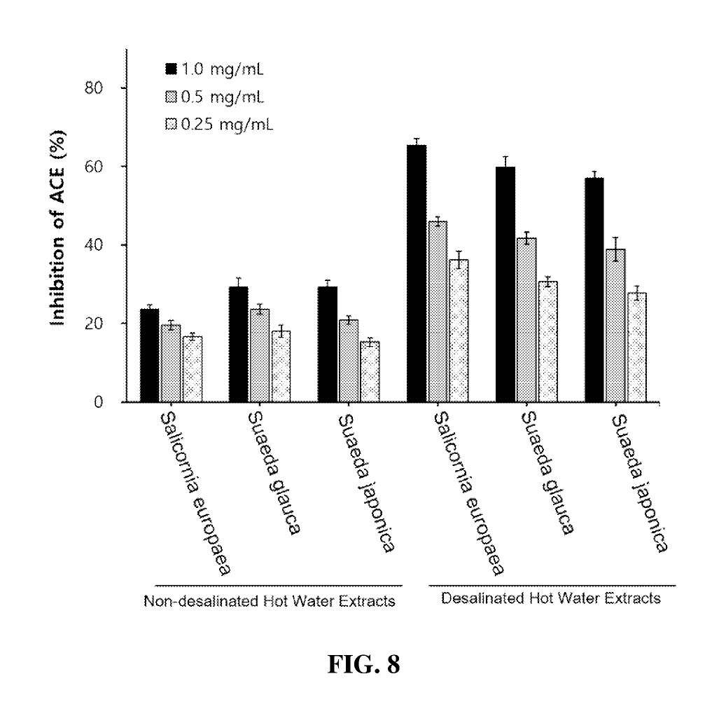

Subsequently, 50 μl of Hip-His-Leu was added as a substrate, and the mixture was allowed to react at 37° C. for 30 min. The reaction was terminated by adding 100 μl of 1 N HCl. Then, 1 ml of ethyl acetate was added, and the reaction mixture was vortexed for 1 min and centrifuged at 3,000 g for 15 min. The separated ethyl acetate supernatant (extract) was recovered in an amount of 0.8 ml. The supernatant was allowed to warm under a hood and was evaporated until dry. When completely dried, it was dissolved in 1 ml of sodium borate buffer. Then, the absorbance was measured at 228 nm, and ACE inhibitory activity was determined. The results are given in FIG. 8. 0 to 1 μg/ml of captopril (Sigma Co., USA) was used as a positive control.

Angiotensin-I-Converting Enzyme (ACE) cleaves the C-terminal dipeptide His-Leu from the decapeptide angiotensin I and thus converts angiotensin I to active angiotensin II, which stimulates blood vessel constriction. The elevated angiotensin II level due to ACE results in a sharp increase in blood pressure. Angiotensin II also stimulates the secretion of the anti-diuretic hormone aldosterone and promotes water and sodium retention by the kidneys, thereby increasing blood volume and blood pressure. In addition, ACE degrades and thereby inactivates bradykinin, which causes blood vessels to relax and thus causes blood pressure to fall, resulting in an increase in blood pressure. The inhibition of ACE activity may prevent vasoconstriction and thus lower blood pressure. Thus, compounds having inhibitory activity against ACE can be developed as a preventive and/or therapeutic agent for hypertension.

As shown in FIG. 8, the halophyte samples prepared in Comparative Example 1 (the non-desalted hot-water extracts) all exhibited, at 1 mg/ml, low ACE inhibitory activity of less than 30%. In contrast, at the same concentration, the halophyte samples prepared in Example 3 (the cold-water-desalted hot-water extracts), at the same concentration, were all found to have remarkably increased ACE inhibitory activity (65.3%, 59.7% and 56.9% inhibition with the samples from S. europaea, S. asparagoides and S. japonica, respectively). These results demonstrated that, when halophyte powder was desalted in cold water for a short time, ACE-inhibiting substances were not eluted, but remained, and were present at high concentrations in the final extract, indicating that the cold-water-desalted hot-water extracts can be used as a functionally reinforced nutritional composition having anti-hypertensive efficacy.

### 3-4: Alpha-Glucosidase Inhibitory Activity

The carbohydrate digestive enzymes maltase, sucrase and glucoamylase are present in the brush border of the small intestine, which are also known as alpha-glucosidases. The inhibition of excessive activities of these enzymes blocks the breakdown of disaccharides and polysaccharides into monosaccharides and thereby delays excessive elevation of blood sugar levels. The inhibition of alpha-glucosidase activity has been used as a tool for measuring anti-diabetic efficacy.

Alpha-glucosidase activity was determined using a slightly modified Ove method (Ove, N.; Cowell, G. M.; Tranum-Jenser, J. Hansen, O.; Welinder, K. G. J. Biol. Chem. 261:12306-12309, 1986).

To a 96-well microplate were added 20 μl of various concentrations (0.25, 0.5 and 1.0 mg/ml) of each sample, 20 μl of alpha-glucosidase (Sigma Co., USA, 2 Unit/ml) and 180 μl of 100 mM phosphate buffer (pH 7.0), followed by preincubation at 37° C. for 10 min. Then, 30 μl of a substrate solution of 20 mM p-nitrophenyl-α-D-glucopyranose was added, and the mixture was allowed to react at 37° C. for 30 min. To evaluate the inhibition of alpha-glucosidase activity, glucose oxidase-peroxidase reagent was added to 180 μl of the reaction mixture in the 96-well plate to generate hydrogen peroxide, which reacts with o-dianisidine to form a colored product. The color intensity was measured at 540 nm, and the absorbance of the reaction mixture was compared with that of a control not containing a sample. As a positive control was used 0 to 10 μg/ml of acarbose (Sigma Co., USA).

Inhibition of alpha-Glucosidase Activity (%)=(1−As/Ac)×100(%)

Ac: 540 nm absorbance of control

As: 540 nm absorbance of sample

Mammalian alpha-glucosidases are digestive enzymes that are present along the brush border membrane of the differentiated enterocytes lining the villi of the small intestine. Alpha-glucosidases stimulate the hydrolysis of dietary carbohydrates in the form of oligosaccharides and polysaccharides into monosaccharides to allow them to be absorbed. Elevated activity of alpha-glucosidase increases such digestion and thus increase the rate of glucose absorption, causing hyperglycemia. Alpha-glucosidase inhibitors delay the digestion of carbohydrates in the small intestine and thereby lower postprandial blood sugar levels while delaying the insulin secretion induced by high blood sugar levels.

Commercially available alpha-glucosidase inhibitors include acarbose, miglitol and voglibose, which have been used for treating type 2 diabetes. Isorhamnetin-β-D-glucopyranoside, which is an antioxidant flavonoid glucoside isolated from an extract of Salicornia europaea, has been reported to have anti-diabetic efficacy.

As shown in FIG. 8, the halophyte samples of Comparative Example 1 (the non-desalted hot-water extracts) exhibited about 15.2 to 40.2% alpha-glucosidase inhibition, while the halophyte samples of Example 3 (the cold-water-desalted hot-water extracts) were found to have remarkably increased inhibitory activity against alpha-glucosidase (70.8%, 76.3% and 65.2% inhibition for the samples from S. europaea, S. asparagoides and S. japonica, respectively). These results demonstrated that, when halophyte powder was desalted in cold water for a short time, alpha-glucosidase-inhibiting substances, which may be present mainly in the form of a flavonoid glucoside and saponin, were not eluted but remained, and were present at high concentrations in the final extract, indicating that the cold-water-desalted hot-water extracts can be used as a functionally reinforced nutritional composition having anti-diabetic efficacy.

### Example 4: Preparation of Salt Substitute Cold-Water-Extracted from Halophytes

100 g of halophyte dried powder (S. europaea, S. asparagoides and S. japonica) was added to 2 liters of cold water (4° C.), stirred at 300 rpm for 4 min, and centrifuged at 10,000 rpm for 20 min. The supernatant having a high salt content was separated while a desalted precipitate was recovered therefrom. The precipitate was further desalted in cold water once more according to the same method as described above, and the second desalted precipitate was recovered. The second supernatant was pooled together with the first supernatant and vacuum-concentrated at 90° C. to achieve a salinity of 18 to 19% and a total solid content of about 26 to 28%. After that, the concentrate was purified using activated carbon in an amount of 5% based on the total solid content, and spray-dried using a spray dryer (EYELA Spray Dryer SD1-1000, Japan), thereby yielding a halophyte-derived cold-water-extracted salt substitute. The cold-water-extracted salt substitute was then evaluated for total salt, cations and glutamic acid content, and the results are given in Table 6 below (the analysis was performed in the research institute of the Korean Food Industry Association).

The cold-water-extracted salt substitute was found, compared to the hot water-extracted salt of Korean Pat. No. 10-0784229, to have low organic content while having high contents of sodium chloride and glutamic acid, which contribute to a clean salty taste having a savory (umami) flavor. In particular, among cations, the sodium to potassium ratio was greater than 10:1 (Na:K), and thus the present cold-water-extracted salt substitute was found to have a high sodium/potassium ratio. These results demonstrated that the cold water extraction for a short time facilitates only sodium, the water solubility of which is not affected by water temperature, unlike other salts, while allowing other cations to remain in the desalted powder (refer to Table 2). Also, the glutamic acid content was found to be remarkably increased compared to that of the conventional hot-water-extracted salt. This result comes from its nature whereby, since glutamic acid is an acidic amino acid that is highly water soluble, it is, unlike other organic substances including other amino acids, easily eluted even under conditions of stirring in cold water for a short time. In addition to this result, it is also a polar compound that is not adsorbed onto activated carbon during purification. Thus, the present invention enables only the sodium chloride component to be effectively removed from halophytes to obtain a functionally reinforced desalted nutritional composition from a halophyte, as well as enabling the residue of desalination to produce a pure vegetable salt having a clean salty taste with a savory (umami) flavor. Therefore, this invention is an innovative method capable of fully (100%) utilizing halophytes.

TABLE 6

| | S. europaea | S. asparagoides | S. japonica |
|---|---|---|---|
| Total salts (%) | 88.6 | 87.4 | 83.8 |
| Na (mg/100 g) | 320,990 | 288,347 | 277,324 |
| K (mg/100 g) | 25,536 | 25,791 | 27.103 |
| Na:K ratio | 12.57:1 | 11.18:1 | 10.23:1 |
| Ca (mg/100 g) | 4.5 | 3.9 | 3.7 |
| Mg (mg/100 g) | 120 | 108 | 125 |
| Fe (mg/100 g) | 0.07 | 0.09 | 0.11 |
| Glutamic acid | 28.50 | 23.18 | 25.30 |
| (mg/g) | | | |

### Test Example 4: Evaluation of Anti-Obesity Effect of the Functionally Reinforced Desalted Nutritional Composition from Halophytes

Referring to Table 2, the total carbohydrate content was found to be increased about 1.85- to 2.06-fold in the desalted nutritional compositions (desalted powder) through desalination of halophytes. These carbohydrates were analyzed and found to be composed of about 95% or greater dietary fiber in S. europaea, S. asparagoides and S. japonica powders. Table 7, below, shows the dietary fiber contents of halophyte powders before and after being desalted. The dietary fiber content includes both soluble and insoluble dietary fiber (the analysis was performed in the research institute of the Korean Food Industry Association).

TABLE 7

| | S. europaea | | S. asparagoides | | S. japonica | |
|---|---|---|---|---|---|---|
| | Before | After | Before | After | Before | After |
| Halophyte | being | being | being | being | being | being |
| powder | desalted | desalted | desalted | desalted | desalted | desalted |
| Dietary | 36.23 | 74.15 | 37.68 | 72.24 | 31.79 | 66.42 |
| fiber | | | | | | |
| content (%) | | | | | | |

Anti-obesity efficacy was investigated for the S. europaea-derived desalted nutritional composition (desalted powder), which has plenty of dietary fiber as well as polyphenols and flavonoids.

### Test Example 4-1: Evaluation for Body-Weight-Reducing Effect of the S. Europaea-Derived Desalted Nutritional Composition in Sprague-Dawley Rats with Obesity Induced by High-Fat Diet

The desalted powder of Salicornia europaea (the desalted nutritional composition), from which 95% or more sodium was removed and which is made rich in dietary fiber, polyphenols and flavonoids through a cold water-desalting process, was evaluated for anti-obesity efficacy. The evaluation was performed in high-fat diet-induced obese Sprague-Dawley (SD) rats using the Desalted Salicornia Powder (DSP) prepared in Example 2, the non-desalted Salicornia Powder (SP) as a comparative control and, as a positive control, the commonly available natural anti-obesity material Garcinia Extract (GE), which is an extract from the roots of Garcinia cambogia. SD rats were randomly divided into five groups each consisting of ten rats, as follows: G1: normal control group, G2: obesity control group induced by high-fat diet, G3: administered with 200 mg/kg of Salicornia Powder (SP), G4: administered with 200 mg/kg of Desalted Salicornia Powder (DSP), and G5: positive control group administered with 200 mg/kg of Garcinia Extract (GE).

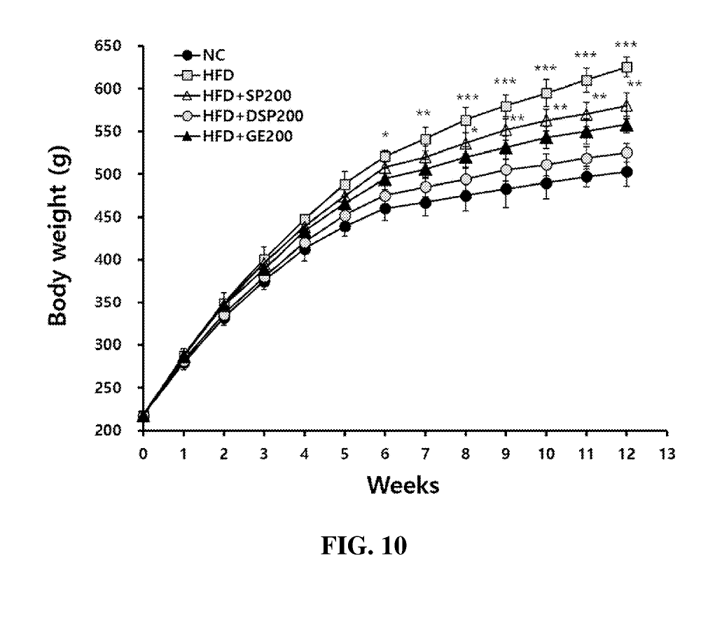

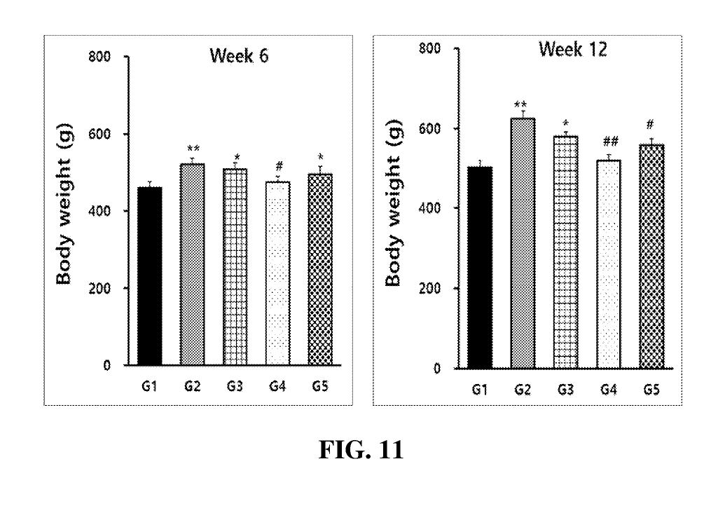

FIG. 10 shows the changes in the average body weight of rats in the five groups over 12 weeks, and FIG. 11 shows the results of statistical analysis of the body weight of rats at 6 and 12 weeks. As shown in FIG. 10, rats in the obesity control group fed with the high-fat diet showed increased body weight starting at three or four weeks compared with the normal control group, and, at 6 weeks, the body weight of the obesity control group was significantly increased compared to the normal control group (p<0.05). The body weight of the group administered with 200 mg/kg of Desalted Salicornia Powder (DSP) was found to be significantly lower than that of the obesity control group (p<0.05). In contrast, at 6 weeks, the body weight of the group administered with 200 mg/kg of non-desalted Salicornia Powder (SP) was found to be significantly higher than that of the normal control group (p<0.05). These results indicate that the Desalted Salicornia Powder (DSP) has a remarkably excellent anti-obesity effect compared to the non-desalted Salicornia Powder (SP).

At 8, 9 and 10 weeks after administration, the obesity control group showed a remarkable increase in body weight compared to the normal control group (p<0.001), and the body weight of the group that received the Desalted Salicornia Powder (DSP) was kept at a significantly low level compared to the obesity control group (p<0.001), while the body weight of the positive control group that received Garcinia Extract (GE) was also significantly low (p<0.05). In contrast, the body weight of rats in the group that received the non-desalted Salicornia Powder (SP) was found to be significantly high compared to the normal control group, in which obesity was not induced (p<0.01), while being reduced compared to that of the obesity-induced control group. At 11 and 12 weeks after administration, the body weight of rats in the obesity control group, the group administered with 200 mg/kg of the non-desalted Salicornia Powder (SP) and the positive control group was significantly high compared to the normal control group (p<0.01 or p<0.05), whereas the body weight of rats in the group administered with 200 mg/kg of Desalted Salicornia Powder (DSP) and the positive control group was significantly low compared to that of the obesity-induced control group (p<0.001 and p<0.01, respectively). Taken together, the body-weight-reducing effect of the test samples in the high-fat-diet-induced obese SD rats, the Desalted Salicornia Powder (DSP) exhibited the most excellent body-weight-reducing effect, which was statistically significantly higher than that of the positive Garcinia Extract (GE) control (p<0.001). In contrast, the non-desalted Salicornia Powder (SP) showed a slight decrease in body weight in the obese rats, but this decrease was remarkably lower than that of DSP. These results are thought to come from the fact that, compared to the desalted Salicornia powder, the non-desalted Salicornia powder was found to have low content of dietary fiber as well as polyphenols and flavonoids, which inhibit lipid synthesis, while having a high content of sodium chloride, and such a composition may act as a potential factor to induce obesity. Therefore, the desalted Salicornia powder, from which sodium chloride was removed and which was enriched in dietary fiber and functional compounds, may be a functional material highly effective for suppressing obesity.

### Test Example 4-2: Evaluation for Body-Fat-Reducing Effect of the S. europaea-Derived Desalted Nutritional Composition in SD Rats Having Obesity Induced by High-Fat Diet

### 4-2-1. Biochemical Blood Test and Body Fat Analysis

12 weeks after obesity was induced, blood samples of about 1 ml were collected from the jugular vein of all rats, injected into a vacutainer tube containing a clot activator, and kept at room temperature for 15 to 20 min to allow coagulation. The blood samples were then centrifuged at 3,000 rpm for 10 min to obtain sera. Thereafter, the sera were analyzed using a blood biochemical analyzer (7020 Hitachi, Japan) for alanine transaminase (ALT), aspartate transaminase (AST), total cholesterol (TC), triglyceride (TG), high density lipoprotein (HDL), low density lipoprotein (LDL) and atherosclerosis index (AI). The results are given in Table 8, below.

TABLE 8

| | NC | HFD | HFD + SP200 | HFD + DSP200 | HFD + GE200 |
|---|---|---|---|---|---|
| TC (mg/dL) | 71.3 ± 2.9 | 151.9 ± 3.5*** | 134.8 ± 5.1**^{#} | 101.6 ± 5.6^{###} | 117.6 ± 4.4*^{##} |
| TG (mg/dL) | 117 ± 7 | 171.2 ± 8.5*** | 139.7 ± 4.6**^{#} | 120.5 ± 6.6^{###} | 125.7 ± 5.8*^{##} |
| HDL (mg/dL) | 28.6 ± 0.7 | 29.1 ± 0.8 | 29.3 ± 1.2 | 30.7 ± 1.2 | 29.7 ± 0.7 |
| LDL (mg/dL) | 8.9 ± 0.9 | 16.6 ± 0.9*** | 13.5 ± 1.0** | 10.9 ± 0.8*^{##} | 12.8 ± 0.6**^{#} |
| VLDL | 17.6 ± 1.5 | 34.2 ± 1.7** | 26.9 ± 0.9*^{#} | 20.1 ± 1.3^{###} | 24.1 ± 1.2^{##} |
| (mg/dL) | | | | | |
| AI | 2.49 ± 0.05 | 5.22 ± 0.07*** | 3.97 ± 0.10**^{#} | 2.90 ± 0.08*^{###} | 3.62 ± 0.08*^{##} |
| AST (U/L) | 129.1 ± 7.2 | 181.8 ± 12.3** | 157.9 ± 9.9* | 139.0 ± 8.0^{##} | 152.3 ± 9.5^{#} |
| ALT (U/L) | 60.1 ± 3.8 | 137.6 ± 4.8*** | 118.7 ± 4**^{#} | 100.1 ± 3.9*^{##} | 114.4 ± 4.6**^{#} |

Data are represented as mean±SD (n=10). Significance of difference between NC group and HFD group and between HFD+SP200, HFD+DSP200 and HFD+GE200 were analyzed by One-way ANOVA and Dunnett's multiple comparisons.

* p<0.05, ** p<0.01, *** p<0.001, ^{#} p<0.05, ^{##} p<0.01, ^{###} p<0.001

As shown in Table 8, at 12 weeks after administration, AST levels of the obesity control group (HFD) induced with the high-fat diet (HFD) and the HFD+SP200 group, which was administered with HFD plus 200 mg/kg of the non-desalted Salicornia Powder (SP) were significantly higher than that of the normal control group (NC) (p<0.01 and p<0.001). The HFD+DSP200 group, which was administered with HFD plus 200 mg/kg of the Desalted Salicornia Powder (DSP), showed a significantly low AST level compared to the HFD group (p<0.01). In the serum levels of TG and TC, the HFD group, the HFD+SP200 group and the positive control group (HFD+GE200) showed a significant increase compared to the normal control group (NC) (p<0.001, p<0.01 and p<0.05), while the HFD+DSP200 group and the positive control group (HFD+GE200) exhibited a significant decrease compared to the HFD group (p<0.001 and p<0.01). In LDL levels, the HFD group, the HFD+SP200 group and the positive control group (HFD+GE200) showed a significant increase compared to the normal control group (NC) (p<0.001 and p<0.01). This tendency was also similarly observed in VLDL and ALT levels. That is, in high-fat-diet-induced obese rats, the administration of the Desalted Salicornia Powder (DSP) was found to markedly reduce the highly increased serum levels of triglyceride (TG), total cholesterol (TC) and LDL, caused by the high-fat diet, as well as the ALT and AST levels elevated by hepatic steatosis, and these decreases were higher than those in the positive control Garcinia Extract (GE). This effect of reducing fat level and ALT and AST levels in blood was also observed in the group administered with the non-desalted Salicornia Powder (SP), but the effect was remarkably low compared to that of the Desalted Salicornia Powder (DSP).

### 4-2-2. Measurement of Abdominal Fat Volume Using Micro-CT

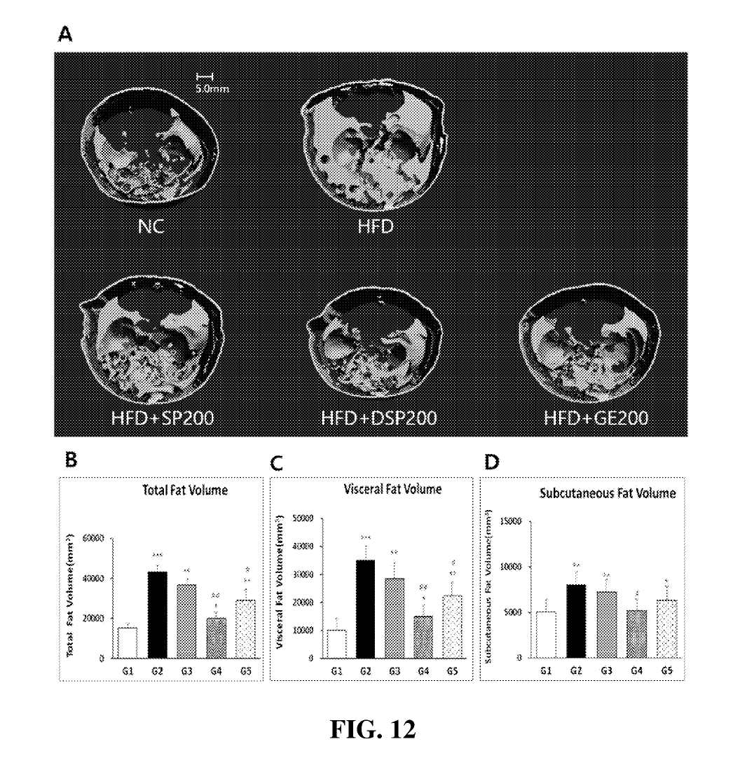

At 12 weeks after obesity was induced, before autopsy, all rats were scanned by Micro-CT (vivaCT 80, SCANCO Medical, Switzerland) to determine abdominal fat volume (FIG. 12A). To measure abdominal fat, scans were taken in the abdominal region (L2-L5) present in the space between the upper edge of the 2nd lumbar vertebra and the lower edge of the 5th lumbar vertebra. At 12 weeks after administration, the total abdominal fat volume was significantly increased in the high-fat-diet-induced obesity control group (HFD) and the HFD+SP200 group administered with the non-desalted Salicornia Powder (SP), compared with the normal control group (NC) (p<0.01 and p<0.05), whereas it was significantly reduced in the HFD+DSP200 group administered with the Desalted Salicornia Powder (DSP) and the positive control group (HFD+GE200) compared with the HFD group (p<0.01 and p<0.05) (FIGS. 12A and 12B).

As shown in FIG. 12C, the visceral fat volume was found to be significantly increased in the HFD group, the HFD+SP200 group and the positive control group (HFD+GE200) compared with the normal control group (NC) (p<0.01), whereas it was significantly reduced in the HFD+DSP200 group compared with the HFD group and the HFD+SP200 group (p<0.01). As shown in FIG. 12D, the subcutaneous fat volume was found to be significantly increased in the HFD group and the HFD+SP200 group compared with the normal control group (NC) (p<0.01 and p<0.05), whereas it was significantly reduced in the HFD+DSP200 group and the positive control group (HFD+GE200) compared with the HFD group (p<0.01 and p<0.05).

### 4-2-3. Statistical Analysis

For the results of the present experiments, parametric One-way ANOVA test was applied under the assumption of the normality of data. The homogeneity of variance was tested by Levene's test. The significant difference between test groups was determined using Duncan's multiple range test where the ANOVA results were significant and variances were equal and using Dunnett's T3 test where variances were unequal. Statistical analysis was done through SPSS Statistics 18.0 K, and values of P<0.05 were considered statistically significant.

Taken together the results of the experiments in high-fat-diet-induced obese SD rats, a) the Desalted Salicornia Powder (DSP) remarkably reduced body weight compared with the obesity-induced control group; b) effectively reduced blood lipid levels (TG, TC, LDL and VLDL), blood ALT and AST levels and atherosclerosis index (AI); and c) as apparent in the results of Micro-CT scanning of tested animals, remarkably reduced the body fat, abdominal fat and subcutaneous fat compared with the obesity-induced control group. As such, the desalted Salicornia powder (DSP) was found to have a remarkably excellent effect of reducing body weight and suppressing body fat accumulation compared with the non-desalted Salicornia powder (SP), and this effect was higher than that of the positive control Garcinia Extract (GE).

### Test Example 5: Determination of a Marker Compound Effective in Suppressing Adipocyte Differentiation in the S. europaea-Derived Desalted Nutritional Composition

5-1. Isolation of a Major Effective Marker Compound from the S. europaea-Derived Desalted Nutritional Composition

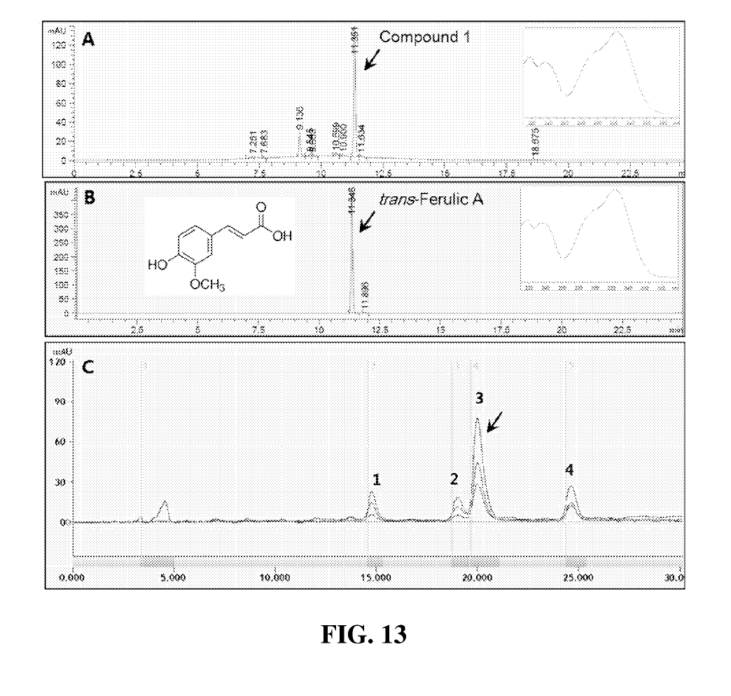

To 100 g of the desalted nutritional composition from Salicornia europaea (desalted powder), prepared according to the method of Example 2, was added 1 L of distilled water and two digestive enzymes, namely amylase and protease. After incubation at 37° C. for 6 hrs, the composition was centrifuged at 10,000 g for 25 min. The supernatant was vacuum-concentrated and freeze-dried. 15.9 g of the sample (DSP-EW) thus obtained through the treatment of the desalted Salicornia powder with digestive enzymes was dissolved in methanol, and methanol-soluble components were then analyzed by high performance liquid chromatography (Agilent HPLC, USA). As shown in FIG. 13A, a major peak compound (Compound 1) was observed near a retention time of 11.3 min. The UV spectrum of this peak component (λmax: 218-220, 240, 285-290sh, 325) showed the typical phenylpropanoid phenolic acid nature. Thus, the HPLC retention time and the UV spectrum were compared with standards of several kinds of phenylpropanoid phenolic acids (Sigma Co., USA). As a result, Compound 1 was identified to be trans-ferulic acid (FIG. 13B).

After that, from 1 g of the methanol-soluble components of the DSP-EW sample, trans-ferulic acid was purified by preparative high performance liquid chromatography (YMC-HPLC, Japan), and used in an experiment with 3T3-L1 cells. The analytical HPLC system used in this experiment was a model 1260 (Infinity, Agilent, USA) equipped with Zorbax Eclipse C18 column (5 μm, 4.5×250 mm, Agilent) and 1200 DAD detector. In the preparative HPLC was used a model (Multiple Preparative HPLC (LC-forte/R, YMC, Japan) equipped with a prep column (Triart C18, 20 mm×150 mm, 5 μm, YMC, Japan). Preparative HPLC was performed under a gradient condition of methanol and triple-distilled water as a mobile phase at a flow rate of 15 ml/min using an YMC UV-3400 UV detector set at three wavelengths, namely 210, 254 and 320 nm, and four fractions were purified. As shown in FIG. 13C, the major peak (compound 3) was found to be trans-ferulic acid, and 230 mg was finally obtained. The other three peaks 1, 2 and 4 were found to be caffeic acid, p-coumaric acid and isorhamnetin-3-β-D-glucoside, respectively.

5-2. The Adipocyte Differentiation-Inhibiting Effect of Trans-Ferulic Acid Purified from the S. Europaea-Derived Desalted Nutritional Composition

5-2-1. Differentiation of 3T3-L1 Preadipocytes

The major marker component of the desalted Salicornia powder, trans-ferulic acid (TFA), was evaluated for its inhibitory effect on adipocyte differentiation using 3T3-L1 cells as an in-vitro model of adipocyte differentiation. 3T3-L1 preadipocytes were checked for contamination every eight hours to improve confidence in the experiment. Primary preadipocytes were propagated, and were then induced to differentiate with a culture medium containing 3-isobutyl-1-methylxanthine (IBMX), dexamethasone and insulin. During the differentiation induction, the medium was changed twice every three days.

5-2-2. Oil Red O Staining and Detection for Intracellular Triglyceride Formation

After being induced to differentiate, 3T3-L1 cells were subjected to Oil Red O staining to detect the presence of intracellular lipid droplets. First, the medium was discarded from each well, and cells were fixed with 4% paraformaldehyde. Subsequently, cells were washed with 100% 1,2-propanediol dehydration solution for 5 min and then stained with Oil Red O stain solution. After Oil Red O staining, 85% 1,2-propanediol stain differential solution was added to each well for cell washing. Finally, distilled water was added to each well to prevent stained cells from drying, and cells were observed under a microscope to determine lipid accumulation.

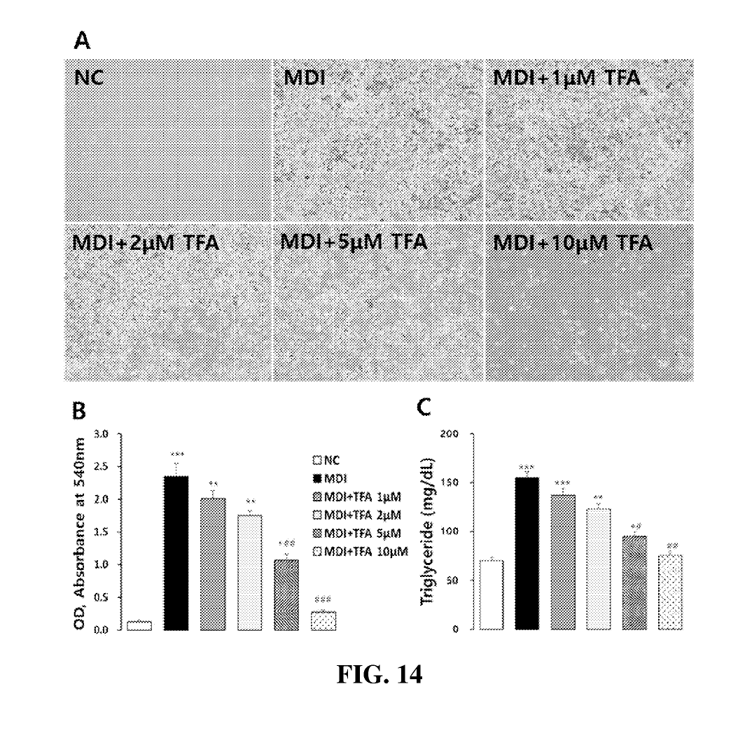

FIG. 14A shows the results of Oil Red O staining to determine the formation of lipid droplets during differentiation of 3T3-L1 preadipocytes to adipocytes so as to investigate the inhibitory effect of trans-ferulic acid (TFA) on adipocyte differentiation. As shown in FIG. 14A, TFA was found to inhibit adipocyte differentiation and lipid droplet formation in a dosage-dependent manner within the used concentrations. In particular, at concentrations of 5 μM and 10 μM, TFA exhibited remarkably significant inhibition compared to an adipocyte differentiation-induced control (MDI) (^{##}p<0.01 and ^{###}p<0.001, see FIG. 14B).

In addition, as a marker for adipocyte differentiation, intracellular triglyceride formation was investigated. As shown in FIG. 14C, TFA reduced intracellular triglyceride formation in a dosage-dependent manner at various concentrations, namely 1, 2, 5 and 10 μM. In particular, at concentrations of 5 μM and 10 μM of TFA, triglyceride formation was remarkably significantly inhibited compared to a differentiation-induced control (MDI) (^{###}p<0.001). These results indicate that TFA inhibits adipocyte differentiation by suppressing differentiation-associated protein expression and eventually reduces lipid synthesis caused by adipocyte differentiation.

5-2-3. Real-Time RT-PCR for Detection of Transcription Factors Involved in Lipid Metabolism

PPARγ, FAS, SREBP-1 and C/EBPα are transcription factors involved in lipid metabolism, and are produced when 3T3-L1 preadipocytes differentiate into mature adipocytes. C/EBPα and PPARγ cooperate to accelerate adipogenesis. When preadipocytes proliferate to an early differentiation stage, C/EBPα is induced and stimulates PPARγ to induce a mature stage of differentiation. PPARγ is present mainly in adipose tissue and regulates overall lipid formation, and its capacity in adipocyte differentiation is much better than other transcription factors. FAS can be used as a marker gene when adipocyte differentiation reaches a late stage, and it is a lipid synthesis enzyme involved in lipid metabolism. FAS is most strongly expressed in adipose tissues, and is the last factor of adipose differentiation. FAS is thus a representative marker for anti-obesity effect, and is induced by SREBP-1, which is a transcription factor at an earlier stage.

Real-Time RT-PCR was performed to investigate the effect of trans-ferulic acid (TFA) on transcription factors involved in lipid metabolism at mRNA expression levels. Total RNA was isolated from a control group and each test group treated with various concentrations of TFA (easy-Blue, iNtRon, INC, Daejeon, Korea) and diluted at the same concentration, and cDNA synthesis was carried out using the diluted RNA samples (cDNA reverse transcription kits, Applied Biosystems, CA, USA). The synthesized cDNA was analyzed for gene expression by Real-Time RT-PCR using the primers listed in Table 9, below.

TABLE 9

| | Primer Sequence | |
|---|---|---|
| Gene | Sense | Anti-sense |
| C/EBPα | SEQ ID NO 1: | SEQ ID NO 2: |
| | 5 CAA CGC AAC GTG | 5 GTC ATT GTC ACT |
| | GAG A-3 | GGT C-3 |
| FAS | SEQ ID NO 3: | SEQ ID NO 4: |
| | 5 GCT GTT GGA AGT | 5 GTT CGT TCC TCG |
| | CAG C-3 | GCG TG-3 |
| PPARγ | SEQ ID NO 5: | SEQ ID NO 6: |
| | 5 GCC CTT TAC CAC | 5 GTT CTA CTT TGA |
| | AGT TGA-3 | TCG CAC TT-3 |
| SREBP-1 | SEQ ID NO 7: | SEQ ID NO 8: |
| | 5 CAG AAG CTC AAG | 5 CAT GCC CTC CAT |
| | CAG GA-3 | AGA CA-3 |
| GAPDH | SEQ ID NO 9: | SEQ ID NO 10: |
| | 5 GGC CTT CCG TGT | 5 GCT TCA CCA CCT |
| | TCC TA-3 | TCT TGA T-3 |

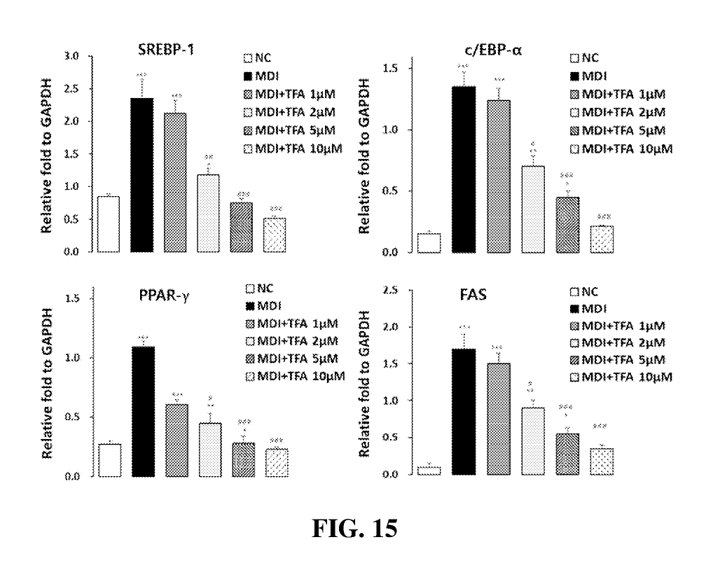

In brief, after 3T3-L1 preadipocytes were treated with TFA at various concentrations, namely 1, 2, 5 and 10 μM), Real-time qRT-PCR was performed using the primers of Table 9 (CFX96 real-time PCR detection system, Bio-Rad Laboratories, Hercules, Calif., USA). The PCR conditions included denaturation at 5° C. for 3 min, 45 cycles of 95° C. for 5 sec and 60° C. for 20 sec, and heating up to 95° C. (by increasing the temperature at a rate of 0.2° C./15 sec) to terminate the reaction. As shown in FIG. 15, FAS and SREBP-1 gene expression was reduced in a dosage-dependent manner compared to a differentiation-induced control (MDI), with the largest decrease at 10 μM of TFA and a considerable decrease at 2 μM and 5 μM of TFA. C/EBPα gene expression was significantly reduced in a dosage-dependent manner at 5 μM and 10 μM of TFA compared to a differentiation-induced control (MDI). Also, PPARγ gene expression was significantly reduced at 10 μM of TFA. Taken together, TFA was found to effectively suppress the gene expression of four transcription factors involved in lipid metabolism, PPARγ, FAS, SREBP-1 and C/EBPα (^{***}p<0.001), thereby inhibiting adipocyte differentiation and lipid accumulation. Therefore, the Desalted Salicornia Powder (DSP) containing TFA as an effective component may effectively reduce body fat by inhibiting adipocyte differentiation and lipid droplet formation and may eventually lead to a decrease in body weight, and thus has the potential to be applied as a functional food and feedstuff for preventing and/or treating obesity.

5-2-4. Statistical Analysis

Data were analyzed using one-way ANOVA analysis, and values of P<0.05 were considered statistically significant.

Hereinbefore, the present invention has been described in detail and with reference to specific examples thereof. Although the preferred embodiments of the present invention have been disclosed for illustrative purposes, those skilled in the art will appreciate that various modifications, additions and substitutions are possible, without departing from the scope and spirit of the invention as disclosed in the accompanying claims. Thus, the actual scope of the present invention will be defined by the appended claims and equivalents thereof.

### INDUSTRIAL APPLICABILITY

The halophyte-derived functionally reinforced desalted nutritional composition can be developed as a pharmaceutical composition and functional food and feedstuff for combating obesity and for reducing body fat.

1. A method of preparing a functionally reinforced desalted nutritional composition from a halophyte, comprising the steps of:
(a) mixing dried powder of the halophyte with water at 9° C. or lower and stirring a resultant mixture;
(b) centrifuging the stirred mixture and removing a supernatant having a high salt content to recover a desalted precipitate; and
(c) drying the desalted precipitate.

2. A functionally reinforced nutritional composition from a halophyte, comprising sodium of less than 6.8 wt % and carbohydrates of 61 wt % or greater, based on a dry weight.

3. The functionally reinforced nutritional composition from the halophyte as set forth in claim 2, comprising potassium (K) of 0.1 to 3.0 wt %, calcium (Ca) of 0.1 to 2.0 wt % and magnesium (Mg) of 0.1 to 1.5 wt %, based on the dry weight.

4. The functionally reinforced nutritional composition from the halophyte as set forth in claim 2, comprising polyphenols of 0.1 to 10.0 wt % and flavonoids of 0.1 to 7.0 wt %, based on the dry weight.

5. The functionally reinforced nutritional composition from the halophyte as set forth in claim 2, comprising chlorophylls of 0.3 to 10.0 wt % based on the dry weight.

6. The functionally reinforced nutritional composition from the halophyte as set forth in claim 2, comprising trans-ferulic acid.

7. A method of preparing a functionally reinforced desalted extract from a halophyte, comprising the steps of:
(a) mixing dried powder of the halophyte with water at 9° C. or lower and stirring a resultant mixture;
(b) centrifuging the stirred mixture and removing a supernatant having a high salt content to recover a desalted precipitate;
(c) extracting the desalted precipitate in a liquid phase to obtain an extract; and
(d) drying the liquid-phase extract.

8. The method of preparing the functionally reinforced desalted extract from the halophyte as set forth in claim 7, further comprising drying the desalted precipitate before the extracting the desalted precipitate in the liquid phase.

9. A functionally reinforced desalted extract from a halophyte, which is extracted from a desalted product of the halophyte and has a total salt content of less than 11.0 wt % and insoluble dietary fiber of less than 3.2 wt %, based on a dry weight.

10. The functionally reinforced desalted extract from the halophyte as set forth in claim 9, comprising polyphenols of 0.1 to 10.0 wt % and flavonoids of 0.1 to 7.0 wt %, based on the dry weight.

11. The functionally reinforced desalted extract from the halophyte as set forth in claim 9, comprising chlorophylls of 0.3 to 10.0 wt % based on the dry weight.

12. A method of preparing a cold-water-extracted salt substitute from a halophyte, comprising the steps of:
(a) mixing dried powder of the halophyte with water at 9° C. or lower and stirring the mixture;
(b) centrifuging the stirred mixture to obtain a supernatant;
(c) concentrating the supernatant and purifying the concentrate with activated carbon; and
(d) spray-drying the purified concentrate.

13. The method of preparing a cold-water-extracted salt substitute from the halophyte as set forth in claim 12, which has a total salt content of 50.0 wt % or more and has a salt composition in which potassium (K) and sodium (Na) are contained at a weight ratio (K:Na) ranging from 1:10.1 to 1:19.0.

14. A cold-water-extracted salt substitute from a halophyte, which has a total salt content of 50.0 wt % or more and has a salt composition in which potassium (K) and sodium (Na) are contained at a weight ratio (K:Na) ranging from 1:10.1 to 1:19.0.

15. The cold-water-extracted salt substitute from the halophyte as set forth in claim 14, comprising glutamic acid in an amount of 0.1 to 50 mg/g.

16. A pharmaceutical composition for combating obesity and for reducing body fat, comprising the halophyte-derived functionally reinforced desalted nutritional composition of claim 2.

17. A functional food for combating obesity and for reducing body fat, comprising the halophyte-derived functionally reinforced desalted nutritional composition of claim 2.

18. A feedstuff for combating obesity and for reducing body fat, comprising the halophyte-derived functionally reinforced desalted nutritional composition of claim 2.

19. A pharmaceutical composition for combating obesity and for reducing body fat, comprising the halophyte-derived trans-ferulic acid of claim 6.

20. A functional food for combating obesity and for reducing body fat, comprising the halophyte-derived trans-ferulic acid of claim 6.

21. A feedstuff for combating obesity and for reducing body fat, comprising the halophyte-derived trans-ferulic acid of claim 6.


SEQUENCE LISTING (10 sequences)

SEQ ID NO: 1 - DNA, 19 bp, Artificial Sequence
  c/EBP alpha primer F
aaacaacgcaacgtggaga

SEQ ID NO: 2 - DNA, 19 bp, Artificial Sequence
  c/EBP alpha primer R
gcggtcattgtcactggtc

SEQ ID NO: 3 - DNA, 19 bp, Artificial Sequence
  FAS primer F
gctgctgttggaagtcagc

SEQ ID NO: 4 - DNA, 20 bp, Artificial Sequence
  FAS primer R
agtgttcgttcctcggcgtg

SEQ ID NO: 5 - DNA, 21 bp, Artificial Sequence
  PPAR gamma primer F
caagccctttaccacagttga

SEQ ID NO: 6 - DNA, 23 bp, Artificial Sequence
  PPAR gamma primer R
caggttctactttgatcgcactt

SEQ ID NO: 7 - DNA, 20 bp, Artificial Sequence
  SREBP-1 primer F
aaccagaagctcaagcagga

SEQ ID NO: 8 - DNA, 20 bp, Artificial Sequence
  SREBP-1 primer R
tttcatgccctccatagaca

SEQ ID NO: 9 - DNA, 20 bp, Artificial Sequence
  GAPDH primer F
catggccttccgtgttccta

SEQ ID NO: 10 - DNA, 22 bp, Artificial Sequence
  GAPDH primer R
cctgcttcaccaccttcttgat